(19) United States
(12) Patent Application Publication
(10) Pub. No.: US 2024/0329056 A1
(43) Pub. Date: Oct. 3, 2024

(54) COMPONENT ASSAY FOR ALZHEIMER'S DISEASE IN A LIVING SUBJECT

(71) Applicant: CASSAVA SCIENCES, INC., Austin, TX (US)

(72) Inventors: Hoau-Yan WANG, Philadelphia, PA (US); George B. THORNTON, Abilene, TX (US)

(73) Assignee: CASSAVA SCIENCES, INC., Austin, TX (US)

(21) Appl. No.: 18/291,014

(22) PCT Filed: Jul. 22, 2022
(86) PCT No.: PCT/US2022/037974

§ 371 Date: Jan. 22, 2024

Related U.S. Application Data
(60) Provisional application No. 63/225,080, filed on Jul. 23, 2021.

(51) Int. Cl. G01N 33/68

(52) U.S. Cl. CPC G01N 33/6896; G01N 2440/14; G01N 2800/2821; G01N 2800/52

(57) ABSTRACT

A method of assaying for the presence of Alzheimer's disease (AD) in a living human subject using a serum or plasma sample preparation from that human subject is disclosed. In one aspect, the presence of an about 90 kDa filamin A (FLNA) polypeptide fragment in a sample preparation indicates that the sample donor likely had AD. More preferably, a ratio of the amount of that about 90 kDa FLNA polypeptide fragment to the amount of full length (about 280 kDa) FLNA protein in the sample preparation is determined. If that ratio is about 10 to about 2000, the donor likely had AD, whereas if that ratio is about 0.005 to about 5, the donor likely did not have AD. A method for determining the treatment prognosis of a living human subject presumed to have Alzheimer's disease (AD), a system and kit for carrying out the assays are also contemplated.

### CROSS-REFERENCE TO RELATED APPLICATION

This application claims priority to U.S. application Ser. No. 63/225,080, filed on Jul. 23, 2021, whose disclosures are incorporated herein by reference.

### GOVERNMENTAL SUPPORT

Research reported in this document was supported by the National Institute On Aging of the National Institutes of Health under Award Number R44AG057329. The government has certain rights in the invention.

### TECHNICAL FIELD

A blood-based assay for Alzheimer's disease in a living subject is disclosed. More particularly, a contemplated assay contemplates the use of a blood component sample such as plasma or serum taken from a living subject in an assay for the presence of Alzheimer's disease in that subject.

### BACKGROUND ART

Alzheimer's disease (AD) represents one of the greatest health care burdens, with 35 million affected individuals worldwide, a population estimated to increase to 115 million by 2050. [Wimo, Alzheimer's Disease International World Report 2010. The Global Economic Impact of Dementia, Alzheimer's Disease International (2010).] AD is a devastating dementia that first presents as progressive memory loss and later can include neuropsychiatric symptoms such as depression, paranoia, agitation and even aggression. Currently, available AD treatment is limited to cognitive enhancers with limited and short-lived efficacy.

Previously, diagnosis of AD could only be confirmed at autopsy by the presence of amyloid deposits and neurofibrillary tangles (NFTs) containing the microtubule-associated protein tau. Current clinical diagnoses of AD satisfy the DSM-IV TR and the NINCDS-ADRDA Work Group criteria for probable AD in McKhann et al., Neurology 34(7):939-944 (1984). Initial diagnostic criteria based mostly on subjective assessments set out in McKhann et al., above, require that the presence of cognitive impairment and a suspected dementia syndrome be confirmed by neuropsychological testing for a clinical diagnosis of possible or probable AD; although they need histopathologic confirmation (microscopic examination of brain tissue) in the postmortem setting for the definitive diagnosis.

The criteria specify eight cognitive domains that may be impaired in AD. Those cognitive domains are: memory, language, perceptual skills, attention, constructive abilities, orientation, problem solving and functional abilities. There are no motor, sensory, or coordination deficits early in the disease. These criteria have shown good reliability and validity, and are those used herein as the basis for assertion of clinical diagnosis of AD.

The diagnosis could not heretofore be determined by laboratory assays. Such assays are important primarily in identifying other possible causes of dementia that should be excluded before the diagnosis of Alzheimer's disease can be made with confidence. Neuropsychological tests provide confirmatory evidence of the diagnosis of dementia and help to assess the course and response to therapy. The criteria proposed by McKhann et al., above, are intended to serve as a guide for the diagnosis of probable, possible, and definite Alzheimer's disease; these criteria will likely be revised as more definitive information become available.

Diagnostic criteria have more recently been refined to include the prodromal phase (early symptoms that occur before the full-blown symptoms of the disease are manifest) termed “Mild Cognitive Impairment (MCI) due to AD.” This new diagnosis reflects a desire to treat the disease earlier because the neuropathology is estimated to start 10 years prior to appearance of symptoms. [Trojanowski et al., Alzheimers Dement 6, 230-238 (2010)]. Clinical trials of potential disease-modifying treatments have been hugely disappointing, possibly in part because even an “early-stage” patient already has a massive amyloid-beta (Aβ) burden and substantial pathologies with significant synaptic defects and inflammation in the key brain regions that regulate cognition.

According to Petersen et al., Arch Neurol 56(3):303-308 (1999), the primary distinction between control subjects and subjects with MCI is in the area of memory, whereas other cognitive functions are comparable. However, when the subjects with MCI were compared with the patients with very mild AD, memory performance was similar, but patients with AD were more impaired in other cognitive domains as well. Longitudinal performance demonstrated that the subjects with MCI declined at a rate greater than that of the controls but less rapidly than the patients with mild AD.

Patients that meet the criteria for MCI can be differentiated from healthy control subjects and those with very mild AD. They appear to constitute a clinical entity that can be characterized for treatment interventions.

Amyloid-beta (Aβ) is a peptide 39-42 amino acid residues in length that is generated in vivo by specific, proteolytic cleavage of the amyloid precursor protein (APP) by β- and γ-secretases. Aβ₄₂ comprises residues 677-713 of the APP protein, which is itself a 770-residue transmembrane protein having the designation P05067 in the UniProtKB/Swiss-Prot system. Aβ, and in particular the Aβ₄₂, is commonly believed to be the principal causative agent in AD, although its mechanism underlying AD neuropathologies is debated.

Until recently, only a symptom-based assay as discussed in McKhann et al., above, was available diagnosing the presence of Alzheimer's disease in a living patient. More recently, beginning in April of 2012 with Eli Lilly's Amyvid®, and later with approval of GE Healthcare's Vizamyl® and Piramal Imaging's Neuraceq®, PET scanning technology has been used to assay for AD in a living human. The intravenous-infused, radiolabeled positron-emitting compound binds to Aβ in brain plaques.

Although accurate, PET scanning assays are inconvenient for patients in that the patients place their heads in a relatively confined space within a scintillation detector and should remain relatively motionless. PET scanning assays are also costly, particularly as compared to a more usual blood test that one receives in which a few milliliters of blood is taken to provide for as many as 40 different assays that unfortunately do not yet commercially include a test for AD.

Recent studies published by several groups have reported initial AD immunoassay results from the amounts of the tau protein phosphorylated at position 181 (tau-p181) present in serum and plasma. See for example, Janelisze et al., Nat Med 26(3):379-386 (Mar. 2, 2020) e-pub Mar. 2, 2020; Karikari et al., Lancet Neurol 19(5):422-433 (May 19, 2020); Thijssen et al., Nat Med 26(3):387-397 (Mar. 2, 2020) e-pub Mar. 2, 2020; and Kasai et al., Mol Neurodegener 12(1):63 (Sep. 4, 2017). Other groups have reported serum-based assays based on Ab. See, Holtzman and Bateman, U.S. Patent Publication 20140370619; Bateman et al., Alzheimer's Dement 16(Suppl 5):e037518 (2020). Each of the above assays is used in conjunction with other assay techniques in the determination of a patient having AD.

Discovered 45 years ago as the first non-muscle actin-binding protein [Hartwig et al., J Biol Chem 250:5696-5705 (1975); Wang et al., Proc Natl Acad Sci USA 72:4483-4486 (1975)], filamins [FLNs] are a family of cytoskeletal proteins—filamins A (FLNA) and B, but not C—that are expressed in non-muscle cells. Human FLNA is given the identifier P21333 in the UniProtKB/Swiss-Prot data base, and contains a sequence of 2647 amino acid residues (about 280 kDa). This protein is also sometimes referred to in the art as actin-binding protein (ABP-280). [Gorlin et al., J Cell Biol 111:1089-1105 (1990).]

The FLNA protein anchors various transmembrane proteins to the actin cytoskeleton and serves as a scaffold for a wide range of cytoplasmic signaling proteins. Filamins are essential for mammalian cell locomotion and act as interfaces for protein-protein interaction [van der Flier et al., Biochim Biophys Acta 1538:99-117 (2001)]. Besides its role in cell motility, FLNA is increasingly found to regulate cell signaling by interacting with a variety of receptors and signaling molecules. [Stossel et al., Nat Rev Mol Cell Biol 2:138-145 (2001); Feng et al., Nat Cell Biol 6:1034-1038 (2004)].

The FLNA protein consists of an N-terminal actin-binding domain (ABD) and a rod-like domain of 24 immunoglobulin-like repeat domains (IgFLNa's) (each about 96-amino acid residues long and numbered from the N-terminus), interrupted by two 30-amino acid residue flexible loops or hinges. The IgFLNa's are numbered 1 through 24, beginning near the N-terminus and ending near the C-terminus. The loop designated H1 is between repeats 15 and 16, and the loop designated H2 is located between repeats 23 and 24 [Gorlin et al., J Cell Biol 111:1089-1105 (1990); van der Flier et al., Biochim Biophys Acta 1538:99-117 (2001)].

H1 and H2 can be cleaved by calpains and caspases [Gorlin et al., J Cell Biol 111:1089-1105 (1990); Browne et al., J Biol Chem 275:39262-39266 (2000)]. Cleavage at H1 occurs between amino acid residues 1762 and 1764, and results in an about 170 kDa fragment consisting of the ABD and repeats 1-15 (IgFLNa-1-15), plus an about 110 kDa polypeptide fragment consisting of repeats 16-24 (IgFLNa-16-24).

It is noted that the UniProtKB/Swiss-Prot data base lists a C-terminus for repeat 15 at position 1740 and a N-terminus for repeat 16 as being located at amino acid residue position 1779. On the other hand, Gorlin et al., above, place the calpain cleavage site between residues 1762 and 1764, whereas Garcia et al., Arch Biochem Biophys 446:140-150 (2006) places that site between residues 1761 and 1762. Similarly, Gorlin et al., above, write that earlier authors (Hartwig et al., J Cell Biol 87:841-848 (1980)] reported the full length FLNA molecule to have a molecular weight of 270 kDa at page 1089 and thereafter as having a 280 kDa protein at page 1093.

Garcia et al. above, reports that calpain cleaves full length FLNA into polypeptide fragments of 180, 100, 90 and 10 kDa, whereas Bedolla et al., Clin Cancer Res 15(3):788-796(2009) report proteolysis fragments of 170, 110 and a 90 kDa fragment cleaved from the 110 kDa fragment. Browne et al., above, reported that the cytotoxic T lymphocyte protease granzyme B, grB, cleaves filamin in concert with the lytic protein perforin, and that filamin is also cleaved in a caspase-dependent manner following ligation to a Fas receptor. Western blots WBs) of dying Jurkat cell lysates identified two caspase cleavage polypeptides from the C-terminal region of FLNA having masses of about 110 and 95 kDa were identified. Purified grB cleaved filamin into several polypeptides including those having masses of about 205, 200 and 110 kDa. Polyclonal rabbit antibodies raised to a fusion protein containing 476 amino acid residues from the C-terminal region of FLNA (positions 2172-2647) were used. Umeda et al. J Biochem 130:535-542 (2001) found somewhat similar results (C-terminal 135, 120 and 110 kDa polypeptide fragments) in U937 monoblastic leukemia and Jurkat human T lymphoblastic cells for proteolysis by caspase-3.

It is noted that IgFLNa-16-24 is said to have an apparent mass of about 110 kDa in Loy et al., Proc Natl Acad Sci, USA, 100(8):4562-4567 (2003). That about 110 kDa polypeptide (IgFLNa-16-24) is further cleaved at H2 by calpain with a longer digestion time to yield an about 90 kDa fragment that contains repeats 16-23 (IgFLNa-16-23) [Gorlin et al., J Cell Biol 111:1089-1105 (1990); van der Flier et al., Biochim Biophys Acta 1538:99-117 (2001)].

Because of the differences in residue positions and some molecular weights for the full length FLNA and its proteolysis fragments reported in the art as discussed above, the full length FLNA molecule and the smaller FLNA cleavage product as having molecular weights of “about” 280 kDa and “about” 90 kDa, respectively.

FLNA promotes orthogonal branching of actin filaments and links actin filaments to membrane glycoproteins. Filamin A is dimerized through the carboxy-terminal repeat (repeat 24) near the transmembrane regions, providing an intracellular V-shaped structure that is critical for function.

Each v-shaped FLNA dimer has two antiparallel self-bound domains 24 forming the apex of the “v”, and the remaining domains stretched out much like beads on a string with each of their N-terminal ABD portions bound to an actin molecule. More recently, it has been reported that C-terminus of the ABD, rod segment 1 (IgFLNa-1-15), forms an extended linear structure without obvious inter-domain interactions. Rod segment 2 (IgFLNa-16-23) assumes a compact structure due to multiple inter-domain interactions in which domains 16-17, 18-19 and 20-21 form paired structures. [Heikkinen et al., J Biol Chem, 284:25450-25458 (2009); Lad et al., EMBO J, 26:3993-4004 (2007).]

Proteolysis of FLNA is regulated in part by its phosphorylation on Ser 2152 (S2152) in repeat 20 (IgFLNa-20), which is reported to render the full-length protein stable and resistant to cleavage. [Gorlin et al., J Cell Biol 111:1089-1105 (1990); Garcia et al., Arch Biochem Biophys 446:140-150 (2006); and Chen et al., J Biol Chem 264(24):14282-14289 (1989).]

Loy et al., Proc Natl Acad Sci, USA, 100(8):4562-4567 (2003), report that a H1 cleavage product containing repeats 16-24 and having a molecular weight of about 100 kDa co-localized with the androgen receptor to the nucleus in prostate cancer cells. Those workers noted that FLNA is generally regarded as a cytoplasmic architectural molecule, and characterized their finding of an additional function of its about 100 kDa polypeptide as is produced by calpain cleavage as a nuclear regulator of the androgen receptor to be “entirely unexpected” (at page 4565).

The about 100 kDa FLNA fragment found in a cellular nucleus is not phosphorylated on pS2152. Indeed, phosphorylation on pS2152 is reported to block the cleavage of full length FLNA by calpain in a prostate cancer line and in platelets. [Garcia et al., Arch Biochem Biophys 446:140-150 (2006); and Chen et al., J Biol Chem 264(24):14282-14289 (1989)].

Wang et al., Oncogene 26:6061-6070 (2007), showed that nuclear localization of FLNA correlates with hormone-dependence in prostate cancer. The non-phosphorylated about 90 kDa fragment (IgFLNa-16-23) migrates to the nucleus of hormone-naïve cells in the androgen-dependent form of the disease. Contrarily, in hormone-refractory androgen-independent prostate tumor cells, FLNA was phosphorylated, preventing its cleavage and nuclear translocation. Those authors and co-workers subsequently showed that not only does prostate cancer metastasis correlate with cytoplasmic localization of FLNA, but that metastasis may be prevented by cleavage and subsequent nuclear translocation of the phosphorylated protein. [Bedolla et al., Clin Cancer Res. 15(3):788-796 (2009).]

As a key regulator of the cytoskeleton network, FLNA interacts with many proteins involved in cancer metastasis, [Yue et al., Cell & Biosci 3:7 (2013)] as well as in many other conditions. Thus, Nakamura et al., Cell Adh Migr. 5(2):160-169 (2011), discuss the history of research concerning FLNA and note that the protein serves as a scaffold for over 90 binding partners including channels, receptors, intracellular signaling molecules and transcription factors.

FLNA also has been implicated in tumor progression. FLNA knockout mice show reduced oncogenic properties of K-Ras, including the downstream activation of ERK and Akt. [Nallapalli et al., Mol Cancer 11:50 (2012).] Many different cancers show high levels of FLNA expression in contrast to low FLNA levels in corresponding normal tissue, including colorectal and pancreatic cancers, [Uhlen et al., Mol Cell Proteomics 4:1920-1932 (2005)] and glioblastoma [Sun et al., Cancer Cell 9:287-300 (2006)].

Inhibition of FLNA expression sensitizes cancer cells to both cisplatin and radiation [Sun et al., Cancer Cell 9:287-300 (2006)], and FLNA deficiency in cancer cells similarly sensitizes them to chemotherapeutic agents [Yue et al., DNA Repair (Amst) 11:192-200 (2012)] and radiation [Yue et al., Cancer Res 69:7978-7985 (2009); Yuan et al., J Biol Chem 276:48318-48324 (2001)]. On the other hand, Jiang et al., Int. J. Biol. Sci. 9:67-77 (2013) report that inhibition of filamin A expression leads to reduced metastasis in nude mice implanted with melanoma and breast cancer cells.

Phosphorylation is recognized as a global regulator of cellular activity, and abnormal phosphorylation is implicated in a host of human diseases, particularly cancers. Phosphorylation of a protein involves the enzymatically-mediated replacement of an amino acid side chain hydroxyl of one or more serine, threonine or tyrosine residues with a phosphate group (—OPO₃⁻²).

Phosphorylation and its reverse reaction, dephosphorylation, occur via the actions of two key enzyme types. Protein kinases phosphorylate proteins by transferring a phosphate group from a nucleotide triphosphate such as adenosine triphosphate (ATP) or guanosine triphosphate (GTP) to their target protein. This process is balanced by the action of protein phosphatases, which can subsequently remove the phosphate group.

The amount of phosphate that is bonded to a protein at a particular time is therefore determined by the relative activities of the particular one or more associated kinase and phosphatase enzymes specific to that protein and to the particular amino acid residue(s) undergoing phosphorylation/dephosphorylation. If the phosphorylated protein is an enzyme, phosphorylation and dephosphorylation can impact its enzymatic activity, essentially acting like a switch, turning it on and off in a regulated manner. Phosphorylation can similarly regulate non-enzymatic protein-protein interactions by facilitation of binding to a partner protein.

Protein phosphorylation can have a vital role in intracellular signal transduction. Many of the proteins that make up a signaling pathway are kinases, from the tyrosine kinase receptors at the cell surface to downstream effector proteins, many of which are serine/threonine kinases.

FLNA is phosphorylated at a number of positions in its protein sequence in both normal and in diseased cells such as cancer cells. For example, the enzyme PAK1 (EC 2.7.11.1) is a protein kinase of the STE20 family that regulates cell motility and morphology. FLNA phosphorylation at position 2152 by PAK1 is required for PAK1-mediated actin cytoskeleton reorganization and for PAK1-mediated membrane ruffling. [Vadlamudi et al., Nat. Cell Biol. 4:681-690 (2002); Woo et al., Mol Cell Biol. 24(7):3025-3035 (2004).] Cyclin B1/Cdk1 (EC:2.7.11.22; EC:2.7.11.23) phosphorylates serine 1436 in vitro in FLNA-dependent actin remodeling. [Cukier et al., FEBS Letters 581(8):1661-1672 (2007).]

The UniProtKB/Swiss-Prot data base entry for human FLNA (No. P21333) lists published reports of the following amino acid residue positions as being phosphorylated under different circumstances: 11, 1081, 1084, 1089, 1286, 1338, 1459, 1533, 1630, 1734, 2053, 2152, 2158, 2284, 2327, 2336, 2414, and 2510. Further, polyclonal and monoclonal antibodies are commercially available from one or more of Abgent, Inc. (San Diego, CA), Abcam® Inc. (Beverly, MA), Bioss, Inc. (Woburn, MA), and GeneTex, Inc. (Irvine, CA) that immunoreact with FLNA that is phosphorylated (phospho-FLNA) at serine-1083, tyrosine-1046, serine-1458, serine-2152, and serine-2522.

The 90 kDa FLNA fragment that can localize to the nucleus and interact with transcription factors includes the subspecies with the serine-2152 residue phosphorylated. However, that previously discussed nucleus-localized about 90 kDa FLNA fragment is free of phosphorylation at serine-2152. Indeed, phosphorylation of FLNA at serine-2152 (pS2152 FLNA) has been reported to protect FLNA from proteolysis to form the about 90 kDa fragment [Garcia et al., Arch Biochem Biophys 446:140-150 (2006); Gorlin et al., J Cell Biol 111:1089-1105 (1990); and Chen et al., J Biol Chem 264(24):14282-14289 (1989)].

The present inventors and their coworkers in their publication Wang et al., J. Neurosci. 32(29):9773-9784 (Jul. 18, 2012), which formed a basis for allowed U.S. application Ser. No. 16/030,494, showed for the first time that the Alzheimer's disease-related ligand Aβ₄₂ activates TLR4 through CD14, which activation requires FLNA. That paper also showed that PTI-125 provides an anti-inflammatory effect by similarly reducing FLNA association with toll-like receptor 4 (TLR4) and preventing cytokine release.” [At page 9774, upper left paragraph.]

### BRIEF SUMMARY OF THE INVENTION

The inventors here have found that an about 90 kDa FLNA polypeptide fragment (IgFLNa-16-23) is present in serum or plasma of a living person with Alzheimer's disease (AD), and is absent from serum or plasma of a person without the disease. Initial studies indicate that this 90 kDa FLNA polypeptide fragment can be detected by antibodies that immunoreact with an epitope that contains the serine residue located at position 2152 of the human FLNA sequence when both phosphorylated and non-phosphorylated. This finding suggests that either or both of the S2152-phosphorylated and S2152 non-phosphorylated polypeptides can be present, and the about 90 kDa mass is the biomarker, independent of its pS2152 phosphorylation state.

It should be remembered that the previously discussed literature [e.g., Wang et al., Oncogene 26:6061-6070 (2007); and Chen et al., J Biol Chem 264(24):14282-14289 (1989)] teaches that this polypeptide is protected against phosphorylation as compared to the full-length molecule, and that the full-length protein is phosphorylated on the serine corresponding to serine 2152. Additionally, that about 90 kDa fragment is not assayed for here in the cell nucleus, nor in the cytoplasm [Loy et al., Proc Natl Acad Sci, USA, 100(8):4562-4567 (2003)], but in the serum or plasma.

The present invention thus contemplates a method of assaying for the presence of Alzheimer's disease (AD) in a living human subject using a serum or plasma sample from that human subject. AD being a disease of the brain and there being little exchange of blood components other than those with a molecular mass less than about 1 kDa between the brain and circulating blood, it is quite surprising that any accurate indicator of the presence of this brain disease could be found in the plasma or serum of the circulating blood stream. It is more surprising still that this accurate marker of AD is the relatively high molecular mass about 90 kDa FLNA polypeptide fragment that can be phosphorylated at a serine residue that corresponds in position 2152 of the full length FLNA.

A contemplated method comprises determining (detecting) the presence or absence of an about 90 kDa polypeptide fragment of FLNA (IgFLNa-16-23) among the other proteinaceous materials in that serum or plasma sample that is usually accomplished using an aqueous serum or plasma preparation rather than serum or plasma themselves as is explained hereinafter. That about 90 kDa FLNA polypeptide fragment can be phosphorylated at FLNA serine2152 (pS²¹³²-90 kDa FLNA or pS²¹⁵²-IgFLNa-16-23).
i) The presence of that 90 kDa FLNA polypeptide fragment (in an amount that is significantly greater than a predetermined background amount) indicates that the human subject from whom the blood sample was obtained likely had AD at the time that sample was obtained.
ii) The absence of that 90 kDa FLNA polypeptide fragment, including an amount that is not significantly greater than a predetermined background amount, indicates that the human subject from whom the blood sample was obtained likely did not have AD at the time the sample was obtained.

The aqueous serum or plasma sample preparation is preferably contacted with paratope-containing receptor molecules that immunoreact with one or both of pS²¹⁵²-90 kDa and S²¹⁵²-90 kDa FLNA polypeptides to form a reaction mixture. That reaction mixture is maintained for a time sufficient for the paratope-containing receptor molecules and the pS²¹⁵²-90 kDa and S²¹⁵²-90 kDa FLNA polypeptides and form an immunoreactant. The presence or absence of the phosphorylated or non-phosphorylated about 90 kDa polypeptide fragment of FLNA is detected from the immunoreactant.

The presence or absence of the 90 kDa FLNA polypeptide fragment is preferably determined after separating the proteinaceous materials present in the sample. The proteinaceous materials present in the aqueous serum or plasma sample preparation are separated into at least two portions prior to the contacting step discussed above, wherein the at least two portions include a first portion that may contain said pS²¹⁵²-90 kDa FLNA polypeptide fragment and a second portion that may contain said about 280 kDa FLNA protein. The proteins present in the sample can be separated at least chromatographically such as by size-exclusion chromatography as well as by affinity binding chromatography, isoelectrically, electrophoretically, ultrafiltration and any other method desired to be used.

One preferred chromatographic separation method is by affinity binding chromatography. In this method, a receptor such as a paratope-containing receptor is linked to a support medium such as a polysaccharide resin like Sepharose® or Sephadex® resins that have been activated with cyanogen bromide or other activating agent. The receptor-linked support medium can be prepared as discussed in Scales et al., J Clin Microbiol 2(4):292-295 (1975) and the citations therein.

Illustrative paratope-containing receptors for use in affinity binding include mouse monoclonal MAB1678 from Chemicon International, Inc., that binds to an epitope sequence near to the FLNA N-terminus and outside of the sequence of the 90 kDa C-terminal calpain cleavage fragment and mouse monoclonal SC-17749 IgG2a antibodies that are specific for an epitope mapping between amino acid residues 9-27 near the N-terminus of FLNA available from Santa Cruz Biotechnology, Inc. With one of these illustrative receptors, the heavier about 280 kDa FLNA protein adheres to the support medium and the about 90 kDa FLNA polypeptide fragment passes through with the eluate.

In another preferred embodiment, the presence or absence of the about 90 kDa FLNA polypeptide is determined using reducing SDS-PAGE western blot analysis with a monomercaptan such as 2-mercapoethanol as the reducing agent to separate the proteinaceous portions of the sample. Once separated, the presence or absence of the 90 kDa FLNA polypeptide can be illustratively determined using a receptor molecule that binds specifically to the 90 kDa FLNA polypeptide. Illustrative receptor molecules are discussed hereinafter.

In a further preferred aspect, the proteinaceous portions are separated by ultrafiltration that separates materials of a molecular mass less than about 100 kDa from the higher molecular weight proteinaceous materials in a diluted aqueous serum or plasma sample preparation. The presence or absence of the 90 kDa FLNA polypeptide is determined using the filtrate (ultrafiltrate), whereas the higher molecular weight proteinaceous materials are in the retentate. Preferably also, the separated proteinaceous portions of the sample are identified by contact with a detection reagent that comprises antibodies or portions thereof that specifically immunoreact with an epitope that includes a serine residue present at FLNA sequence position 2152 to form an immunoreactant, although other antibodies and portions thereof that immunoreact with other epitopes of the IgFLNa-16-23 can also be used.

In another preferred embodiment, the presence or absence of the 90 kDa FLNA polypeptide is determined using a sandwich assay such as an ELISA assay. In one preferred aspect, receptors that bind to two different sites on the about 90 kDa FLNA polypeptide are utilized. Preferably, one of those receptors contains a paratope that immunoreacts with an epitope that includes the phosphorylated or non-phosphorylated serine residue present at FLNA sequence position 2152 to form an immunoreactant. In this assay, the proteinaceous materials present in the sample can be separated prior to contacting with the receptors, or that contacting can be carried out without prior separation of the proteinaceous materials.

In a more preferred embodiment, a ratio is determined of the amount of the lower molecular weight, about 90 kDa FLNA polypeptide fragment (A), to the amount of a second proteinaceous material (B) present in an aqueous serum or plasma sample preparation that comprises serum or plasma sample obtained from the living human subject. A particularly preferred second proteinaceous material (B) is the about 280 kDa FLNA protein that can also be phosphorylated on the serine of sequence position 2152 (pS²¹⁵²-FLNA) or not so phosphorylated, although albumin, alyceraldehyde 3-pchosphate dehydrogenase (GAPDH) or another protein present in the subject's serum or plasma and therefore in the aqueous serum or plasma sample preparation can be used. Use of that ratio appears from initial data to tighten and provide more reliable results in correlating different subject populations with different conditions from each other.

When an amount of either A or B is present in a quantifiable amount (QAmt) and the other is not present in a quantifiable amount, an arbitrary predetermined fractional amount that is about 0.1 to about 0.001 of the quantifiable amount is assigned [(0.1-0.001)×(QAmt)] to the unquantifiable other amount to avoid use of zero in a numerator or denominator. When both of A and B are not present in a quantifiable amount, an arbitrary amount is assigned that is about the same for both.

When A is the about 90 kDa FLNA polypeptide fragment and B is the about 280 kDa FLNA protein, that A/B ratio is about 10 to about 2000, the subject likely had AD at the time the sample was obtained, whereas when that ratio is about 0.005 to about 5, the subject likely did not have AD at the time the sample was obtained.

These two ratios are very different in magnitude because those subjects with AD have high quantities of the about 90 kDa FLNA polypeptide and little if any of the about 280 kDa FLNA protein in their serum or plasma. On the contrary, those subjects that are free of the disease have little to none of the lower molecular mass, about 90 kDa FLNA, polypeptide and large amounts of the full-length protein in their serum or plasma. Ratios prepared using a (B) proteinaceous material other than the about 280 kDa FLNA protein amount will differ from those values using filamin A itself, but the values are readily calculated.

Determination of the above-discussed ratios can be carried out in each of the assay formats disclosed herein. Weight or molar amounts of the two proteinaceous materials can be used, but it is usually more convenient to utilize relative amounts as determined by means of colorometric, radioisotopic, fluorescence, or phosphorescence emission labeling techniques as are well known.

Another embodiment of the invention is an assay system for detecting whether or not the about 90 kDa FLNA polypeptide is present in a sample of blood plasma or serum, and thereby the likelihood that the subject from whom the sample was obtained had or did not have AD at the time the sample was obtained. A contemplated system includes a solid phase support whose assay surfaces are coated with paratope-containing capture receptor molecules that immunoreact with an epitope that includes anti-FLNA (receptor molecules); and b) a container holding first detection receptor molecules that bind to and capture the about 90 kDa FLNA polypeptide fragment, when present, to form a captured complex, and a label for indicating the presence of that captured complex such that the presence or absence of the captured complex correlates with presence or absence of the about 90 kDa FLNA polypeptide fragment in the sample. Preferably, the first detection receptors are paratope-containing molecules that immunoreact with phosphorylated or non-phosphorylated serine residue present at FLNA sequence position 2152-serine residues (anti-pS or anti-S receptors).

In preferred aspects, a further container is included that holds second detection receptor molecules that react with a binding site present in full length FLNA that is not present in an about 90 kDa FLNA polypeptide. A label for indicating the presence of that binding is also included.

In preferred practice, the system is a kit in which the recited elements are present packaged together. Instructions for using those receptor molecules for the purpose of binding to the about 90 kDa FLNA polypeptide to form an antibody-antigen complex are also preferably included in the kit.

Illustrative solid phase supports include multi-well plates that permit detection of the about 90 kDa FLNA polypeptide in multiple samples, individual test tubes and particulate solids such as plastic beads and magnetic particles.

A method for determining the prognosis of treatment of a living human subject presumed to have Alzheimer's disease (AD) with a treating compound or a pharmaceutically acceptable salt of that treating compound is also contemplated. That method includes determining the presence of a first amount of an about 90 kDa polypeptide fragment of filamin A (FLNA) in a first aqueous serum or plasma sample preparation comprising a serum or plasma sample obtained from that living human subject. The living human subject is treated with a therapeutic composition containing an anti-AD effective amount of a treating compound or a pharmaceutically acceptable salt of that compound. A second amount of an about 90 kDa polypeptide fragment of FLNA in a second aqueous serum or plasma sample preparation from the human subject is taken at a time at least about one month after the beginning of the treatment. The amount of the about 90 kDa polypeptide fragment of FLNA present in sample preparations of serum or plasma obtained from the blood of the living patient before and after the treatment is determined, wherein a later-determined amount that is significantly less than an earlier-determined amount is consistent with a prognosis of a benefit through use of the treatment to the patient from whom the samples were obtained.

In one preferred aspect, the amounts of about 90 kDa polypeptide fragment of FLNA present in the first and second sample preparations are determined as ratios relative to the amount of a second proteinaceous material that is present in human serum or plasma such that when the ratio of those two proteinaceous materials are compared before and after treatment as recited. Again, a later-determined ratio amount that is significantly less than an earlier-determined ratio amount is consistent with a prognosis of a benefit through use of the treatment to the patient from whom the samples were obtained. Illustrative second proteinaceous materials include albumin, glyceraldehyde 3-phosphate dehydrogenase (GAPDH), and the about 280 kDa FLNA protein. Use of the about 280 kDa FLNA protein is particularly preferred for use in the ratio determination. Illustrative treating compounds areaducanumab and simufilam, or a pharmaceutically acceptable salt of simufilam. Use of simufilam or its pharmaceutically acceptable salt is particularly preferred.

The present invention has several benefits and advantages.

Salient among these benefits and advantages is the fact that accurate results for the presence or absence of AD can be obtained using a relatively unobtrusively-obtained serum or plasma sample.

A salient advantage of this invention is that the determination of the presence or absence of AD is made on a living person that may be treatable for AD or another disease.

A further benefit of the invention is that there is little inconvenience for the patient undergoing the assay as compared to undergoing a PET scan.

A further advantage of the invention is that there is little inconvenience for the assay laboratory carrying out the assay in that the techniques needed are common in the industry and also from the fact that the assay can be performed in a multiplexed, automated manner.

Still further benefits and advantages of the invention will be apparent to the skilled worker from the discussion that follows.

### BRIEF DESCRIPTION OF THE DRAWINGS

In the drawings forming a portion of this disclosure,

FIG. 1, FIG. 2 and FIG. 3 are photographic copies of western blot (WB) analyses of 1.25 μl samples of human plasma from study subjects showing full-length, about 280 kDa FLNA and a FLNA polypeptide portion having a molecular mass of about 90 kDa (90 kDa FLNA). Separations were carried out in the laboratories of inventor H-Y Wang at CUNY School of Medicine, Manhattan, New York, under denaturing conditions on 7.5% SDS-PAGE after boiling diluted samples for 5 minutes in Laemmli SDS-PAGE sample preparation buffer at pH 7.5 that contained 2-mercaptoethanol as reductant and cooling. Following electrophoretic transfer of the separated samples to nitrocellulose, the proteinaceous materials were visualized first by reaction with rabbit anti-human pS-2152-FLNA monoclonal antibodies (Abcam® #75978) and then using HRP-conjugated anti-rabbit IgG and chemiluminescent visualization on X-ray film to provide relative density value numerical amounts of each protein visualized. Albumin and glyceraldehyde 3-phosphate dehydrogenase (GAPDH) were visualized similarly after binding of detection receptors (here, rabbit IgG antibodies) specific to each protein. Numerals beneath the darkened protein bands labeled GAPDH are subject identification numbers. Protein density measurements were also determined for albumin and GAPDH present in the plasma for each indicated subject. Specifics of this method are provided hereinafter.

FIG. 4, FIG. 5 and FIG. 6 are scatter plots of the visualization data of FIG. 1, FIG. 2 and FIG. 3 arrayed by previously determined likely Alzheimer's disease status, in which AD=those with AD (typically determined by Amyvid® assay), AMC=age-matched controls (without AD), YCI=young cognitively intact persons, MCI AD=moderately cognitively impaired AD patients, and MCI NonAD=moderately cognitively impaired persons without AD. FIG. 4 shows data for the about 90 kDa FLNA protein alone, FIG. 5 shows data for the full length phosphorylated FLNA, and FIG. 6 shows data for the ratio of data for about 90 kDa FLNA polypeptide/about 280 kDa FLNA protein (A/B).

FIG. 7 is a photographic copy of western blot analyses of 1.0 μl samples of human plasma from study subjects showing full-length FLNA about 280 kDa and a FLNA polypeptide portion having a molecular mass of about 90 kDa as in FIGS. 1-3, except that protein density measurements were not also determined for albumin and GAPDH present in the plasma. These studies were carried out for the inventors at Altogen Labs, Austin, Texas, using samples provided by Dr. Wang.

FIG. 8 is a graph of the ratio 90 kDa FLNA/FLNA obtained from the density values obtained by scanning the western blots of FIG. 7.

FIG. 9 is a photographic copy of western blot analyses similar to those of FIG. 7 but using different patient samples.

FIG. 10 is a graph of the ratio obtained from the density values obtained by scanning the western blots of the about 90 kDa FLNA polypeptide/about 280 FLNA protein of FIG. 9.

FIG. 11 is a graph showing relative amounts of about 90 kDa FLNA polypeptide fragment and full length (about 280 kDa) FLNA protein using plasma from several subjects whose AD status was not known when the assays were run. Here, the plasma samples were diluted 1:40 with pH 7.4 Tris buffer and split into two portions (unfiltered-light bars; filtered-dark bars). One portion was ultrafiltered through a Nanosep® 100 K OMEGA™ (P/N OD100C33) centrifugal filter (PALL Corp., Ann Arbor, MI) the resulting filtrate and the unfiltered portion were separately contacted with the walls of Reacti-Bind™ NeutrAvidin™ high binding capacity coated 96-well plate walls that had been coated with biotinylated antibodies that specifically react with rabbit monoclonal antibodies to the phosphorylated serine 2152 of FLNA as capture molecules. The captured pS²¹⁵²-about 90 kDa FLNA polypeptide fragment was detected using mouse anti-phosphoserine monoclonal antibodies [NeuroMab (UC Davis/NIH): Cat #: 73-292], whereas the captured pS²¹³²-FLNA was detected using mouse monoclonal IgG2a antibodies specific for an epitope mapping between amino acid residues 9-27 near the N-terminus of FLNA (SC-17749, Santa Cruz Biotechnology, Inc.). The relative amount of each immunoreactant was determined by reaction of each with FITC-labeled anti-mouse antibodies followed by excitation of the fluorescent label and measurement of the relative fluorescence intensities.

FIG. 12A and FIG. 12B are photographic copies of western blot analyses of samples of human plasma from the same study subjects using two different receptors that bind to different epitopes. The receptors of FIG. 12A are rabbit polyclonal antibodies that are phospho-specific for binding to phosphorylated serine 2152 of FLNA available from OriGene, Inc. under the designation TA313881. The receptors of FIG. 12B are mouse monoclonal antibodies raised to a 10-mer N-acetyl-terminated polypeptide corresponding in sequence to positions 2148 through 2157 of the FLNA sequence that includes phosphorylated serine 2152, but the antibodies are not phospho-specific as are the OriGene antibodies. The letters within each column have the following meanings: AD=Alzheimer's patient; MCI-SNAP=MCI suspected non-AD pathology; MCI-AD=MCI with AD pathology; EC=elderly control; YCI=young cognitively intact control; and AD+=Amivid® positive AD patient.

FIG. 13 is a graph showing the amounts in arbitrary units of the about 90 kDa FLNA polypeptide fragment found in the plasma of 13 AD patients in a Phase 2a clinical trial who were treated with a medicament twice daily for 28 days and had their blood plasma assayed on days 0, 14 and 28 for the presence and amount of the about 90 kDa FLNA polypeptide fragment. The data of the graph show the amount of the about 90 kDa FLNA polypeptide fragment present lessened in the group at day 14 versus day 0, and at day 28 versus day 14, indicating that the medicament was effective in its treatment.

FIG. 14A and FIG. 14B are graphs of antibody binding data to a peptide or the recombinant about 90 kDa FLNA polypeptide fragment bound to a solid phase support in an ELISA format provided by microtiter plate wells. The bound human FLNA UniProtKB/Swiss-Prot data base P21233 sequences contained an additional C-terminal cysteinamide for binding to maleimide-activated keyhole limpet hemocyanin (KLH) as the immunogenic carrier for the immunogen. The serine residue at position 2152 of the polypeptide labeled “pS2152” was phosphorylated. The bound antigen in each depicted assay was from right to left: the immunogenic polypeptide, the immunogenic polypeptide in which the phosphorylated serine residue was replaced by an alanine residue (“A2152”), and the 90 kDa polypeptide FLNA fragment whose serine2152 was phosphorylated (“90 kDa FLNA”). FIG. 14A provides data for mouse monoclonal antibody preparation diluted 1:1000 using commercially available (Origene) and two monoclonal antibodies (20H7 an IgG2 and 16E6 and IgM) prepared at the inventors' direction by Aragen Biosciences, Inc., Morgan Hill, CA. FIG. 14B provides binding data using rabbit polyclonal antibodies to the same three targets using antibodies from the third bleed from the numbered rabbits at a 1:200 dilution.

FIG. 15 is a scatter plot of data from Table 1, hereinafter, in which density values for the about 90 kDa polypeptide fragment of FLNA, Albumin, glyceraldehyde-3-phosphate dehydrogenase (GAPDH) and the about 280 kDa FLNA protein values are plotted for serum samples from patients previously determined to have Alzheimer's disease, AD, and age-matched controls, AMC, known to be free of AD. The horizontal black bars indicate the mean±standard error of the mean (SEM) values as paired by the connected horizontal and vertical black lines.

FIGS. 16A, 16B and 16C are further replottings of the data from Table 1 in which the ratios of the density value of the about 90 kDa polypeptide fragment of FLNA divided by the density value for the Albumin (FIG. 16A), or divided by the density value for the GAPGH (FIG. 16B), or divided by the density value for the about 280 kDa FLNA protein for the several subjects are shown for the AD subjects, age-matched control (AMC) subjects, young cognitively intact (YCI) subjects, subjects with mild cognitive impairment due to AD (MCI-AD), and subjects with mild cognitive impairment not due to AD (MCI-nonAD).

### DEFINITIONS

In the context of the present invention and the associated claims, the following terms have the following meanings:

The articles “a” and “an” are used herein to refer to one or to more than one (i.e., to at least one) of the grammatical object of the article.

By way of example, “an element” means one element or more than one element.

“Blood serum” or “serum” is the clear liquid that separates from the blood when blood is permitted to clot completely. It is therefore blood plasma from which fibrinogen has been removed in the process of clotting. [Dorland's Illustrated Medical Dictionary, 29^{th} ed., W.B. Saunders Co. Philadelphia, PA, p. 1629 (2000).]

“Blood plasma” or “plasma” is the clear, straw-colored liquid portion of blood that remains after red blood cells, white blood cells, platelets and other cellular components are removed from un-clotted blood. It is the single largest component of human blood, comprising about 55 percent, and contains water, salts, enzymes, antibodies and other proteins. [The Practice of Medicinal Chemistry, C. Wermuth ed., Academic Press, New York, page 46 (1996).]

A “serum or plasma sample” is an aliquot of serum or plasma that is obtained from the subject after the minimal processing discussed above.

A serum or plasma sample is typically too viscus for use in an assay described herein and is therefore typically diluted with water or an aqueous buffer or other aqueous composition to form a “serum or plasma sample preparation”. Illustrative dilutions are typically about 10- to about 100-fold, and more usually about 20- to about 50-fold. A diluting composition can also contain one or more organic solvents that are compatible with the proteinaceous components of a serum or plasma sample.

The FLNA molecules of interest here have apparent molecular masses (weights) determined by electrophoresis as being “about 90 kDa” and “about 280 kDa”. Those two FLNA molecules are usually referred to herein as the “about 90 kDa FLNA polypeptide fragment” and the “about 280 kDa FLNA protein”. The word “about” is sometimes omitted herein for ease in expression, as are one or more of the words “FLNA polypeptide fragment” and “FLNA protein” with the understanding that the skilled worker will understand that those masses are approximate due to their method of determination.

As noted earlier, according to designation P21333 in the UniProtKB/Swiss-Prot data base, FLNA contains a sequence of 2647 amino acid residues and has a molecular mass of about 280 kDa. Each of those amino acid residues has a specific position number as counted from the amino-terminus of the protein. Even when the protein is cleaved enzymatically, the positional relations of the residues to each other in a resulting polypeptide chain remains the same.

Consequently, it is convenient to refer to particular residues by their UniProtKB/Swiss-Prot data base position numbers whether that residue is in the intact (full=length) protein or a polypeptide portion of the protein. Thus, the serine at position 2152 in the complete FLNA molecule is also referred to as residue 2152 in the about 90 kDa FLNA polypeptide portion of the FLNA molecule; i.e., S²¹⁵²-90 kDa FLNA. When that serine residue is phosphorylated and present in the 90 kDa polypeptide, it is referred to as pS²¹⁵²-90 kDa FLNA. When phosphorylated and present in full-length FLNA, it is referred to as pS²¹⁵²-FLNA.

It is further noted that that the “full length” and “90 kDa polypeptide portion” or “90 kDa polypeptide fragment” refer to those proteinaceous materials as recovered from the person whose body sample is to be analyzed. Those materials are thus intended to include sampled materials that are processed during or after their recovery to change their respective molecular weights.

It is also to be understood that the full length FLNA and 90 kDa polypeptide FLNA fragments are themselves present in the serum or plasma without lysis of cells that may also be present in the blood such as lymphocytes that may occur during recovery of the blood, plasma or serum.

As used herein, the term “receptor” is broadly used to refer to an entity with which another entity, a ligand, specifically binds. A receptor is generally a macromolecule and a ligand is generally a smaller, lower molecular weight molecule, but that distinction is not required. Receptor molecules contemplated herein include whole antibodies and antibody combining site portions (paratopes) that immunoreact with specific epitope ligands, as well as proteins such as Staphylococcus aureus proteins A and G that bind to Fab and Fc antibody portions. Biotin and avidin (streptavidin) can also be viewed as a ligand-receptor pair with either molecule being the receptor or ligand, as can aptamers.

A receptor molecule of the present invention can be an antibody, a substantially intact antibody in substantially purified form, such as is found in ascites fluid or serum of an immunized animal, or an idiotype-containing polypeptide portion of an antibody such as Fab and F(ab′)₂ antibody portions as are described hereinafter. Antibody receptor molecules can be monoclonal and polyclonal antibody receptors, sometimes referred to herein as “monoclonal receptors” or “polyclonal receptors”.

Biological activity of a receptor molecule is evidenced by the specific binding of the receptor to its ligand upon their admixture in an aqueous medium, at least at physiological pH values and ionic strengths. Preferably, the receptors also bind to the ligand within a pH value range of about 5 to about 9, and at ionic strengths such as that of distilled water to that of about one molar sodium chloride.

The words “bind” and “binding” are used herein to be a shortened phrase for specific binding as in an immunoreaction that occurs between an antibody and its antigen, an enzyme and its substrate binding or biotin and avidin binding. Specific binding as above is compared to non-specific binding such as that in which a protein in solution indiscriminately sticks to the plastic or glass walls of a microtiter plate as by hydrophobic or ionic or other non-specific means.

Paratope-containing molecules or polypeptide portions (antibody combining sites) of antibodies are those portions of antibody molecules that specifically bind to an epitope of a ligand, and can include the Fab, Fab′ and F(ab′)₂ portions of the antibodies. Fab and F(ab′)₂ portions of antibodies are well known in the art, and are prepared by the reaction of papain and pepsin, respectively, on substantially intact antibodies by methods that are well known. See for example, U.S. Pat. No. 4,342,566 to Theofilopolous and Dixon. Fab′ portions of antibodies are also well known and are prepared by the reduction of F(ab′)₂ disulfide bonds as by admixture with mercaptoethanol followed by alkylation of the reduced cysteine residues with a reagent such as iodoacetamide. Intact antibodies are preferred receptors, and are utilized as illustrative of the receptor molecules of this invention.

Suitable monoclonal antibody receptors, typically whole antibodies, can be prepared using hybridoma technology described by Niman et. al., Proc. Natl. Sci., U.S.A., 80:4949-4953 (1983), which description is incorporated herein by reference. Briefly, to form the hybridoma from which the monoclonal receptor is produced, a myeloma or other self-perpetuating cell line is fused with lymphocytes obtained from the spleen of a mammal hyperimmunized with a selected proteinaceous immunogen.

It is preferred that the myeloma cell line be from the same species as the lymphocytes. Typically, a mouse of the strain 129 GlX⁺ is the preferred mammal. Suitable mouse myelomas for use in the present invention include the hypoxanthine-aminopterin-thymidine-sensitive (HAT) cell lines P3X63-Ag8.653 (ATCC CRL 1580), and Sp2/0-Ag14 (ATCC CRL 1581). Splenocytes are typically fused with myeloma cells using polyethylene glycol (PEG) 1500. Fused hybrids are selected by their sensitivity to HAT medium. Hybridomas producing useful receptor molecules can be identified using the enzyme linked immunosorbent assay (ELISA).

Illustrative monoclonal receptors include the mouse anti-human FLNA IgG1 monoclonal designated MAB1678 and the mouse anti-human FLNA IgG1 monoclonal designated MAB1680 that are commercially available from Chemicon International, Inc. Mouse monoclonal MAB1680 binds to an epitope sequence within the about 90 kDa FLNA polypeptide C-terminal calpain cleavage fragment, whereas mouse monoclonal MAB1678 binds to an epitope sequence nearer to the FLNA N-terminus and outside of the sequence of the 90 kDa C-terminal calpain cleavage fragment. Additional monoclonal receptors useful herein include the mouse anti-phosphoserine NeuroMab clone N259/48 available from the UC Davis/NIH NeuroMab Facility at UC Davis, Davis, CA; and the rabbit monoclonal anti-pS²¹⁵²filamin A receptors [EP2310AY] Abcam® (cat #: ab75978).

Monoclonal receptors need not only be obtained from hybridoma supernatants, but can also be obtained in generally more concentrated form from ascites fluid of mammals into which the desired hybridoma has been introduced. Production of monoclonal antibodies using ascites fluid is also well known and will not be dealt with further herein. Both MAB1678 and MAB1680 are produced in ascites.

A “polyclonal receptor” (pAb) is a receptor produced by clones of different antibody-producing cells that produce antibodies to a plurality of epitopes of the immunizing molecule. Illustrative polyclonal antibodies useful herein include rabbit pAb sc 28284, whose epitope is said to be at FLNA C-terminal amino acid residues 2348-2647; rabbit pAb sc 130190, whose epitope is said to include FLNA's phosphorylated serine2151; and goat pAb SC 7565, whose epitope is said to map near the N-terminus of FLNA from Santa Cruz Biotechnology, Inc.

In addition to the mice, rabbits and goats noted above, non-human, warm blooded animals usable in the present invention as hosts in which monoclonal or polyclonal receptors are raised can include poultry (such as a chicken or a pigeon), a member of the ratite bird group (such as an emu, ostrich, cassowary or moa) or a mammal (such as a dog, cat, monkey, pig, cow, horse, guinea pig, or rat). Preferably, the host animal is a rabbit or mouse.

It is noted that depending on the assay format, one or two receptor molecules can be used. A first, as used in an ELISA or other solid phase assay, is referred to as the capture receptor molecules, or more simply, capture molecules. Capture molecules are typically themselves bound to a solid phase support and bind to the analyte ligand that is present in a liquid phase such as FLNA or the phosphoproteinaceous FLNA portion having a molecular mass of about 90 kDa that includes amino acid residue position 2152 (pS²¹³²-90 kDa FLNA). A second type of receptor molecule is referred to herein as a detecting receptor molecule, or analyzing molecule. That second receptor binds to the analyte ligand in its bound form and is used for quantifying the amount of ligand captured.

Detecting molecules are utilized along with an indicator labelling means or “indicating group” or a “label”. The indicating group or label is utilized in conjunction with an analyzing molecule as a means for determining that a specific ligand has bound to the capture molecule. An illustrative is a labeled paratope-containing molecule that binds to a genus-specific antibody Fc portion, such as FITC-labeled goat anti-mouse antibodies (Sigma-Aldrich or Abcam®), and HRP-conjugated anti-rabbit IgG (Santa Cruz Biotechnology or GE Lifesciences).

The terms “indicator labelling means”, “indicating group” or “label” are used interchangeably herein to include single atoms and molecules that are linked to a receptor or used separately, and whether those atoms or molecules are used alone or in conjunction with additional reagents. Such indicating groups or labels are themselves well-known in immunochemistry.

The indicator labelling means can be a fluorescent labelling agent that chemically binds to antibodies or antigens without denaturing them to form a fluorochrome (dye) that is a useful immunofluorescent tracer. Suitable fluorescent labelling agents are fluorochromes such as fluorescein isocyanate (FIC), flourescein isothiocyanate (FITC), dimethylaminonaphthalene-S-sulphonyl chloride (DANSC), tetramethylrhodamine isothiocyanate (TRITC), lissamine rhodamine B200 sulphonyl chloride (RB 200 SC) and the like. A description of immunofluorescence analysis techniques is found in DeLuca, “Immunofluorescence Analysis”, in Antibody As A Tool, Marchalonis et al., Eds., John Wiley & Sons, Ltd., pp. 189-231, (1985), which is incorporated herein by reference.

The indicator labelling means can be linked directly to an analyzing molecule or can comprise a separate molecule. It is particularly preferred that the indicator means be a separate molecule such as antibodies that bind to a receptor of this invention. Staphylococcus aureus protein A, sometimes referred to herein as protein A, can also be used as a separate molecule indicator or labelling means where an intact or substantially intact antibody receptor is utilized. In such uses, the protein A itself contains a label such as a radioactive element or a fluorochrome dye.

The indicating group can also be a biologically active enzyme, such as horseradish peroxidase (HRP) or glucose oxidase, or the like. Where the principal indicating group is an enzyme such as HRP or glucose oxidase, additional reagents are required to visualize the fact that a receptor-ligand complex has formed. Such additional reagents for HRP include hydrogen peroxide and an oxidation dye precursor such as diaminobenzidine. An additional reagent useful with glucose oxidase is 2,2′azino-di-(3-ethylbenzthiazoline-6-sulfonic acid) (ABTS).

More modern technology utilizes luminol as an oxidation precursor that chemiluminescences when oxidized as with a HRP-hydrogen peroxide reaction product. Illustrative chemiluminescing reagents for use in conjunction with HRP include those sold under the trademark names SuperSignal® ELISA Pico Chemiluminescent Substrate by ThermoFisher Scientific, Waltham, MA; westernSure® ECL Substrate sold by LI-COR Biosciences of Lincoln, NE; PicoMax™ Sensitive Chemiluminescent HRP Substrate, sold by Rockland Immunochemicals, Inc., Limerick, PA. The chemiluminescent assays are understood to be more accurate and precise than a colorimetric assay, as well as exhibiting enhanced signal to noise and greater sensitivity in a sandwich ELISA format assay. See for example, Hatch et al., J. Med. Biol Sci 3(1):1-6 (2009) and Wang et al., J Bioprocess Biotech 3(2):136 (2013).

HRP-Conjugated goat anti-rabbit IgG polyclonal antibodies that are useful as an indicating group are available from several suppliers. Illustrative materials include product 12-348 from EMD Millipore; ab6721 from Abcam®; product 65-6120 from ThermoFisher Scientific; and product sc-2030 from Santa Cruz Biotechnology.

Radioactive elements provide another class of label. An exemplary radio-labelling agent that can be utilized in the invention is a radioactive element that produces gamma ray emissions. Elements which themselves emit gamma rays, such ¹²⁴I, ¹²⁵I, ¹²⁸I, ¹³¹I, ¹³²I, and ⁵¹Cr represent one class of gamma ray emission-producing radioactive element indicating groups. Particularly preferred is ¹²⁵I.

Another class of useful indicating groups are those elements such as ¹¹C, ¹⁸F, ¹⁵O, and ¹³N that themselves emit positrons. The positrons so emitted produce gamma rays upon encounters with electrons present in the analysis medium. Also useful is a beta ray emitter, such as ¹¹¹indium.

A radioactive receptor molecule can be made by culturing receptor-producing cells in a medium containing radioactive amino acids, as is well known, as well as by isolating the receptor and then labelling the receptor with one of the above radioactive elements. Radiolabeling of proteins is well known in the art and will not be discussed further herein.

Receptor molecules and separate indicating means of any diagnostic kit described herein can be provided in solution, as a liquid dispersion or as a substantially dry powder, e.g., in lyophilized form. Where the indicating means is a separate molecule from the analyzing receptor, it is preferred that the indicating means be packaged separately. Where the indicating means is an enzyme, the enzyme's substrate can also be provided in a separate package of the kit. A solid support such as a microtiter plate, one or more buffers and other desired reagents can also be included as separately packaged elements in this diagnostic assay kit.

The phrases “system” and “kit” are used herein with slightly different meanings. A “system” includes recited elements and a “kit” can contain the same elements. The difference between the two is that in a kit, those elements are packaged together, whereas a system just uses the elements, regardless of their source.

The packages discussed herein in relation to diagnostic kits are those customarily utilized in diagnostic systems. Such packages include glass and plastic (e.g., polyethylene, polypropylene, polystyrene and polycarbonate) bottles, vials, plastic and plastic-foil laminated envelopes and the like.

As used herein, the phrase “significantly different” and “significant difference” and like terms and phrases mean that if a difference between two or more findings is found, on repeating that assay enough times to obtain a statistically reliable result, the compared results differ by greater than one standard deviation of either measurement, preferably by greater than two standard deviations, and more preferably by three standard deviations.

Each of the patents, patent applications and articles cited herein is incorporated by reference. The use of the article “a” or “an” is intended to include one or more.

### DETAILED DESCRIPTION OF PREFERRED EMBODIMENTS

The present invention contemplates a method of assaying for the likely presence of Alzheimer's disease (AD) in a living human subject using a serum or plasma sample from that human subject using the relatively non-invasive technology of blood sampling. In another aspect, a contemplated assay can be used as a basis for an objective prognostic and biomarker assay that can indicate the prognosis of treatment as well as to track disease progression and treatment efficacy in a living, presumed AD patient.

AD patient plasma has been found to contain the 90-kDa pS²¹⁵²FLNA fragment with no 280-kDa pS²¹⁵²FLNA protein. We hypothesize that the 90-kDa pS²¹⁵²FLNA fragment is derived from degenerated and ruptured neurons in the brain. To assess this hypothesis, an ex vivo organotypic brain slice culture was used that exhibits AD-like pathologies and neurodegeneration following protracted Aβ₄₂ exposure [Wang et al., Biol Psychiatry 67(6):522-530 (2010)].

Using two brain regions known to be vulnerable to AD damage, 200-μm sections of prefrontal cortex and hippocampus were prepared from 10-week-old rats. These brain slices were cultured as described previously (Wang et al., above) for 4 days to permit recovery of slice preparation damage. These slices were then serum-deprived and incubated with 100 μM Aβ₁₋₄₂ (Aβ₄₂).

The presence of the 90-kDa pS²¹⁵²-FLNA fragments and pS²¹⁵²-FLNA protein, as well as other biomarkers that reflect neuronal destruction, nitrated tau (nY²⁹-tau) and glial damage [truncated glial fibrillary acidic protein (GFAP)] in the culture medium were assessed by Western blotting. The specificity of Aβ₄₂ was defined by Aβ₄₂₁, an intrareversal peptide.

The 90-kDa pS²¹⁵²-FLNA fragment, but not full-length (280-kDa) pS²¹⁵²-FLNA protein, appears in the medium starting at 4 hours post-Aβ₄₂ addition and steadily increases over time. The time-course is comparable with the progression of nY²⁹-Tau, suggesting that the 90-kDa pS²¹⁵²-FLNA fragment originates from neuronal (axonal) damage. In addition, full-length GFAP, followed by its truncated form, appears early following Aβ₁₋₄₂ exposure.

These data suggest that glial damage precedes neuronal destruction. Further, there was more prominent axonal/neuronal damage than that observed for glial damage in hippocampal tissues compared to prefrontal cortex. These data indicate that Aβ₄₂ inflicts destruction to both neurons and glia.

Although FLNA is expressed in both neurons and glia, the time-course of appearance of 90-kDa pS²¹⁵²-FLNA in the medium suggests degenerated neurons as the primary source. The absence of 280-kDa pS²¹⁵²-FLNA protein in the slice medium coupled with elevated 280-kDa pS²¹⁵²-FLNA protein levels in AD postmortem brains suggests that the 90-kDa pS²¹⁵²-FLNA fragment detected in AD plasma is mainly derived from destroyed neurons and is the result of protease digestion of the 280-kDa pS²¹⁵²-FLNA protein prior to its release into plasma. These data also support the notion that Aβ₄₂ can be a causative agent of AD pathology.

Based on the brain slice data, the full-length pS²¹⁵²FLNA in cognitively normal elderly in plasma most likely originates in the periphery. Platelets are a likely source because they are known to have large quantities of FLNA.

Thus, it has been found that a subject's plasma or serum that contains an about 90 kDa FLNA polypeptide fragment or an about 90 kDa FLNA polypeptide fragment that is phosphorylated on the serine of FLNA residue position 2152 (pS²¹⁵²-90 kDa FLNA) correlates positively to subjects that have AD. To the contrary, absence of an about 90 kDa FLNA polypeptide fragment from a subject's serum or plasma sample correlates with the subject not having AD.

Those correlations, while generally correct, can sometimes provide inaccurate conclusions when based solely on the presence or absence in serum or plasma of the about 90 kDa FLNA (pS²¹³²-90 kDa or S²¹⁵²-90 kDa FLNA) protein fragment (polypeptide), or the about 280 kDa FLNA protein. The possibility of inaccurate conclusions can be particularly the case with older subjects, such as those individuals older than about 45 years of age, and particularly with subjects about 60 years old and older.

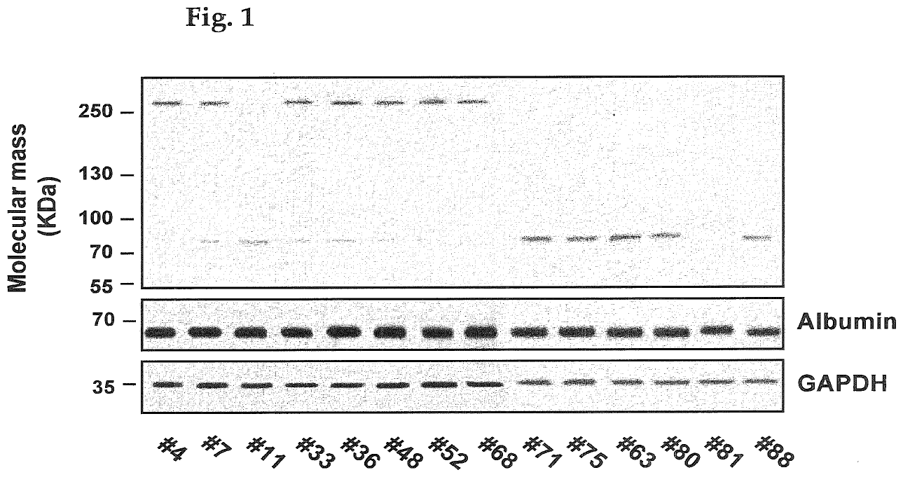

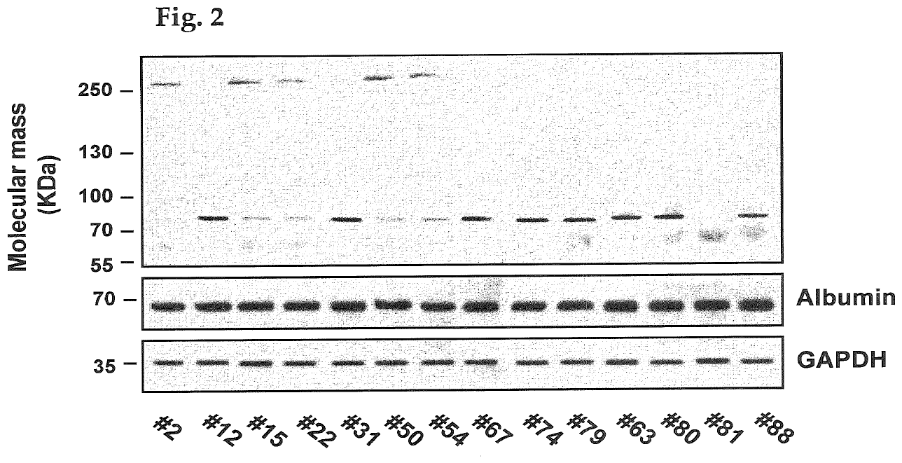

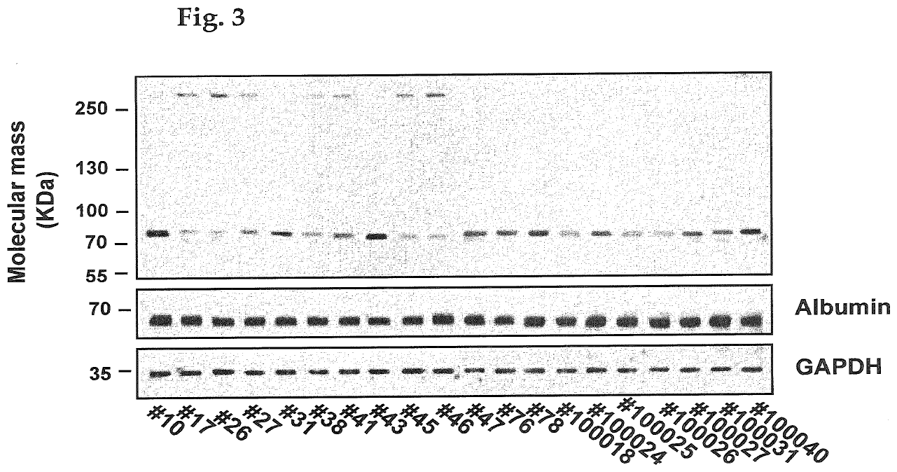

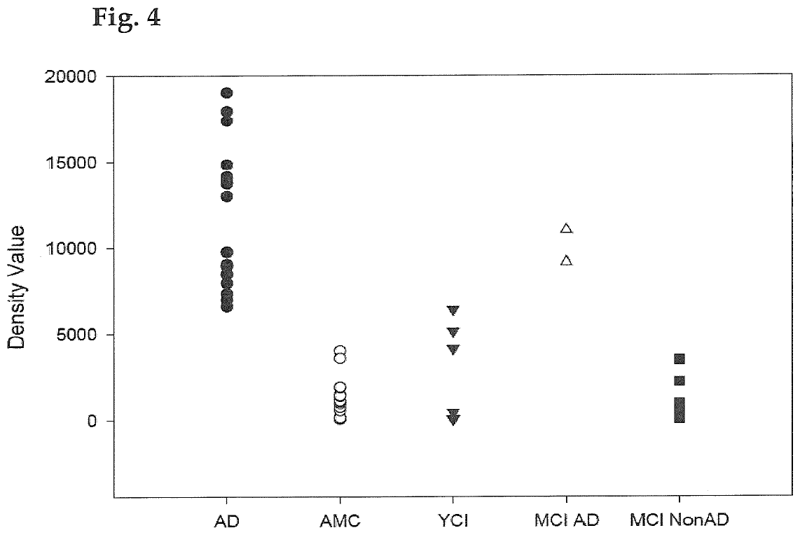

Examination of FIGS. 4 and 5 shows the scatter of data points obtained when one plots the individual density values obtained from the data in FIGS. 1, 2, and 3. However, when one examines the ratio of the density value from a subject's sample preparation for the about 90 kDa FLNA polypeptide fragment divided by the value for the about 280 kDa FLNA protein as are shown in FIG. 6, the data spread is lessened, while there is still no overlap between the subjects with (AF and MCI AD) and without AD (AMC and MCI NonAD).

Thus, amounts of both low [about 90 kDa FLNA] and high molecular mass [about 280 kDa; presumably intact FLNA] whose position 2152 serine residue is phosphorylated or not, as discussed below, are preferably used together in a ratio to provide a more accurate assessment of the presence or absence of AD in the subject that provided the blood sample used as of the time of donation. Data for the young control intact (YCI) subjects spread somewhat, but those subjects rarely have AD. Although AD patient serum contains the about 90 kDa FLNA fragment, it is not known whether all of the 90 kDa FLNA fragment present in serum or plasma originates in the AD brain, or in some other tissue or both places.

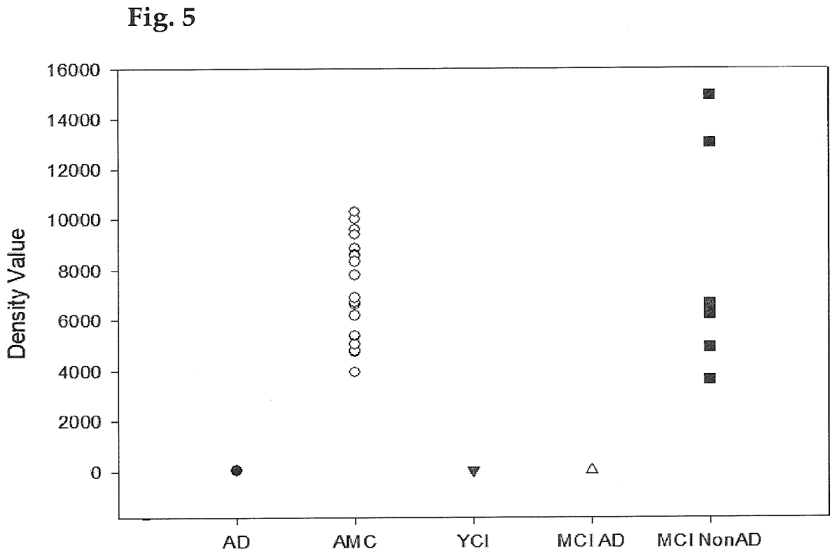

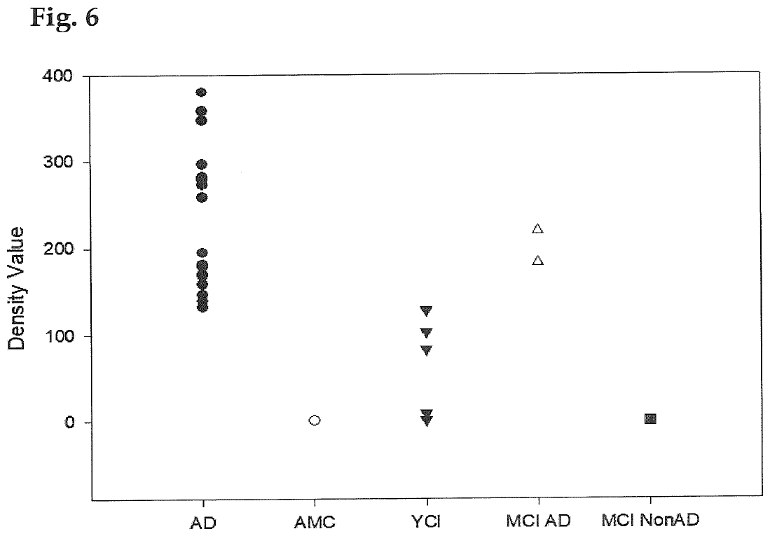

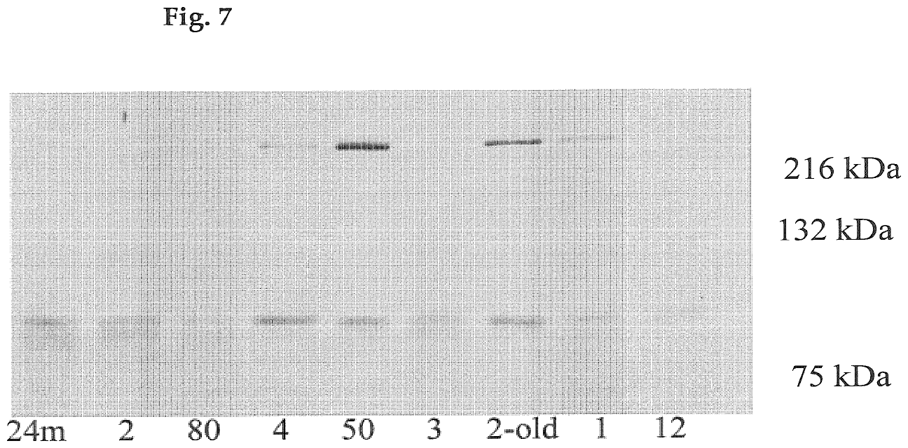

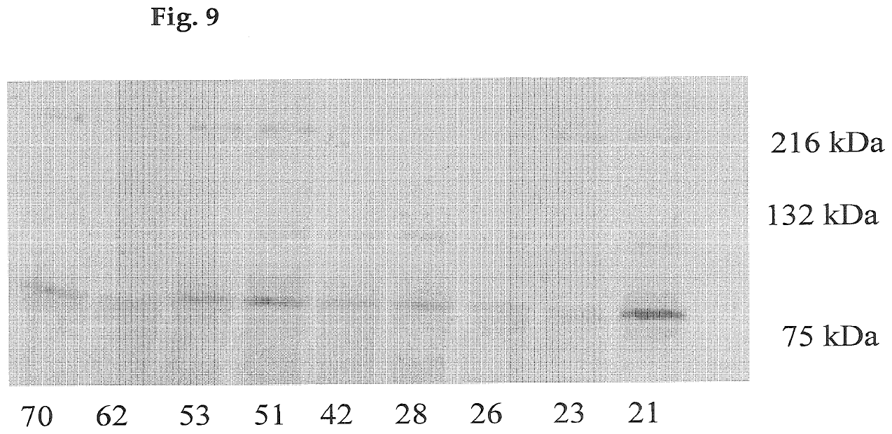

In one preferred embodiment, the presence or absence of phosphorylated or non-phosphorylated S²¹⁵²-90 kDa FLNA and/or S²¹⁵²-FLNA is determined using reducing SDS-PAGE western blot separation and analysis. A monomercaptan such as 2-mercapoethanol is used as the sample reducing agent prior to separating the proteinaceous portions of the sample. Illustrative western blot analyses of several human serum or plasma sample preparations prepared from plasma serum or samples are shown in the visualized separations illustrated in FIGS. 1-3, 7 and 9.

The underlying data shown in Table 1, discussed hereinafter in the Results section, show that the upper values for the young cognitively intact (YCI) subjects and the age-matched controls (AMC) approach but do not overlap with the lower values of AD patients. The data of Table 1 for the ratio of (about 90 kDa polypeptide fragment)/(about 280 kDa FLNA protein) and the scatter plot of FIG. 6 show that there is no overlap when a ratio of amounts of the two FLNA-related phosphorylated proteinaceous materials is used as discussed herein. This separation is especially relevant for the AMC population. Similar results are obtained using receptors that do not immunoreact with a non-phosphorylated serine2152 residue.

It was unexpected to find an about 90 kDa FLNA polypeptide fragment in the plasma or serum of presumed AD subjects. The present inventors are not aware of any report of the presence and/or detection of this phosphorylated about 90 kDa FLNA polypeptide fragment (pS²¹⁵²-90 kDa FLNA) or any about 90 kDa FLNA polypeptide fragment in the serum or plasma of anyone. That polypeptide fragment has previously only been reported to appear in a cell's nucleus, as was noted earlier.

Further, an antibody specific for binding to an epitope containing the phosphorylated serine at FLNA position 2152 was used to identify the species shown in FIGS. 1-6 so that the about 90 kDa FLNA polypeptide fragment of those figures is phosphorylated at the serine of protein position 2152, and possibly at other presently undefined positions. Not only is that blood-born about 90 kDa FLNA polypeptide fragment species phosphorylated, it is phosphorylated at the serine residue corresponding to serine 2152 in the full length about 280 kDa FLNA protein molecule that otherwise had been shown in the art [Garcia et al., Arch Biochem Biophys 446:140-150 (2006); Gorlin et al., J Cell Biol 111:1089-1105 (1990)] to inhibit proteolytic processing of the FLNA leading to the presence of full-length phosphorylated (pS2152) FLNA and an about 90 kDa FLNA polypeptide fragment that is not phosphorylated at serine2152 in the nucleus in prostate cancer cells.

The about 90 kDa FLNA polypeptide fragment species can usually be easily distinguished in a western blot analysis from the full-length FLNA whether those molecules are phosphorylated at serine 2152 or not. As a result, receptor molecules that immunoreact with epitopes containing phosphorylated serine 2152 as well as receptors that immunoreact with epitopes not containing phosphorylated serine 2152 can be utilized in this assay.

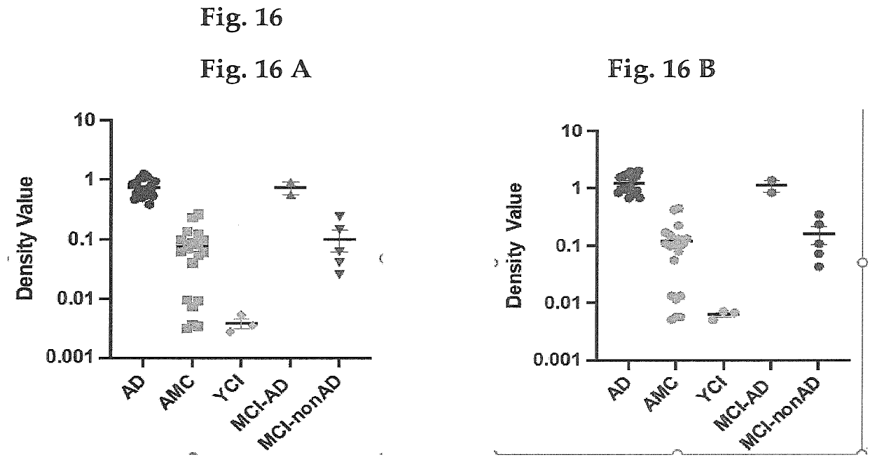

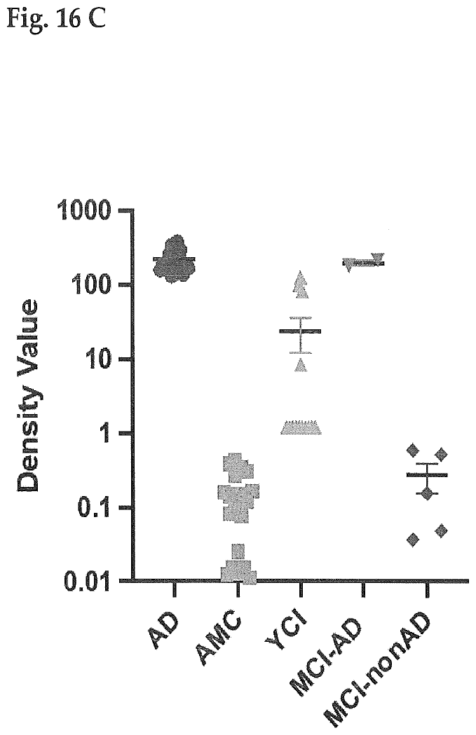

It was even more unexpected that the amount of a phosphorylated about 90 kDa FLNA (pS²¹⁵²FLNA) polypeptide or of an about 90 kDa FLNA polypeptide not phosphorylated at serine 2152 species present in a blood plasma or serum sample could be used with the amount of another protein to accurately diagnose the AD status of the subject that provided the plasma or serum sample. Thus, the data of FIGS. 16A, 16B and 16C that are discussed in greater detail hereinafter, show that the ratio of amounts (densities) of an about 90 kDa FLNA polypeptide to the amount (density) of another proteinaceous material present in the same aqueous plasma or serum sample preparation can be used to reduce the scatter of amount of the about 90 kDa FLNA polypeptide alone to produce a viable assay. Illustrative other sample preparation proteinaceous species illustrated are albumin, glyceraldehyvde 3-phosphate deldrogenase (GAPDH) and the about 280 kDa FLNA protein. Also surprisingly, the about 280 kDa FLNA protein proved to be the most useful ratio partner thus far found.

It is to be understood that although use of a ratio of an about 90 kDa FLNA polypeptide fragment or the about 280 kDa FLNA protein to another proteinaceous material of the same subject's serum or plasma is preferred, use of either of those materials alone can also yield an assay that provides the correct answer. Consequently, although the assays discussed herein will generally be discussed in terms of use of a ratio, those individual materials can also be used separately.

A contemplated assay can also be used in conjunction with a treatment method for AD to indicate advancement, stasis or regression of the disease state. Thus, an increase in the ratio of about 90 kDa FLNA polypeptide fragment/about 280 kDa FLNA protein, or of either of their 2152-phosphorylated counterparts, indicates advancement of the disease (worsening), whereas a decrease in the ratio indicates a lessened progression of disease, and no change in the ratio indicates no change in the disease status.

In one aspect, a contemplated assay is preferably carried out in a western blot format following a SDS-PAGE separation under reducing conditions as is illustrated and discussed hereinafter. The SDS-PAGE separation in which the protein is denatured under reducing conditions uses a monomercaptan reducing agent.

2-Mercaptoethanol is preferred and usually used as the reducing agent, although thioglycolic acid and thioglycolate derivatives such as glyceryl monothioglycolate, ethyleneglycol monothioglycolate, thioglycolamide (2-mercaptoacetmide), or a C₁-C₆-hydrocarbyl thioglycolate can be used. The use of reducing agents having two or more mercapto (—SH) groups (polymercapto compounds) such as dithiothreitol (DTT) or dithioerythritol (DTE) has been found to interfere with the assay, and a reducing composition used in this assay is free of such polymercapto reducing agent compounds.

Once electrophoretically separated, separated proteinaceous portions are then transferred to a membrane such as nitrocellulose or polyvinylidene difluoride (PVDF) and bound to or reacted with identifying reagents, as discussed hereinafter and is well known in the art.

Continuing with the steps of a method, a detection reagent is contacted with the first separated FLNA fraction (e.g., the about 90 kDa FLNA polypeptide fragment) to form a first detection reagent-bound FLNA fraction. If that lower molecular mass polypeptide is present, it is likely that the subject had AD at the time the sample was taken. Ultrafiltration of a diluted aqueous serum or plasma preparation using an appropriate ultrafilter, can provide the about 90 kDa FLNA polypeptide fragment in the ultrafiltrate (filtrate) as is also illustrated herein.

More preferably, a detection reagent is contacted with the second, higher molecular weight separated FLNA fraction (e.g., about 280 kDa FLNA protein) to bind the detection reagent to the second separated FLNA fraction. When each of the about 90 kDa FLNA polypeptide fragment and the about 280 kDa FLNA protein is present, first and second detection reagent-bound FLNA fractions are formed. It is preferred that the same detection reagent be used to contact both phosphorylated FLNA species. Use of the same detection reagent for both phosphorylated FLNA species can remove avidity and affinity differences between two different receptor molecules thus simplifying calculations.

The amount of the detection reagent-bound FLNA first fraction (A) and the detection reagent-bound FLNA second fraction (B) is quantified. The quantification step is usually carried out densitometrically, by fluorimetry or where a radiotracer is used, by radio-disintegrations using a commercially available apparatus designed specifically for western blot analyses.

The amount of detection reagent-bound material present can sometimes be determined in an actual amount in grams or moles. More usually, the amount present is determined relative to an internal standard or as an amount that is greater than a background amount. These methods of relative amount determinations are well known in the art.

Thus, in one preferred assay, a ratio of the amounts of the above-discussed lower and higher molecular mass polypeptide and protein, respectively, is utilized. It is currently preferred that the ratio be that of the amount of the lower molecular weight polypeptide to the amount of the higher molecular weight protein be used. In one aspect, this determination is also preferably carried out as a western blot assay following SDS-PAGE protein separation using a monomercaptan as the reducing agent.

A ratio of the amounts of the two phosphorylated proteins is preferred for at least two reasons. First, with the exception of “young cognitively intact” (YCI) subjects, who would be unlikely to be examined in a contemplated assay, almost everyone whose serum or plasma is examined will have a small amount one or both of the polypeptide and/or protein. Second, providing two assayable proteinaceous materials to determine a ratio can provide a control to the assay in that if too much or too little sample is used in the assay or if insufficient receptor molecules are used, the use of two values in a ratio can minimize an error.

Contrary to that usual practice, in the present assays, one or both of the assayed FLNA-related molecules can be absent or not present in a quantifiable amount. When an amount of either the low molecular weight polypeptide (A) or higher molecular weight (B) protein is present in a quantifiable amount (QAmt) and the other is not present in a quantifiable amount, an arbitrary predetermined fractional amount that is about 0.1 to about 0.001 of the quantifiable amount is assigned [(0.1−0.001)×(QAmt)] to the unquantifiable other amount to avoid use of zero in a numerator or denominator. When both of A and B are not present in a quantifiable amount, an arbitrary amount is assigned that is about the same for both. Where one of A and B is present in a quantifiable amount and the other is not, that present amount is typically in the thousands of relative units such as density units and an arbitrary amount of about 30 to about 70, and preferably about 50 or 60, can be used for the unquantifiable amount.

When a ratio of polypeptide/protein amounts is used in a preferred assay, with the low molecular weight amount in the numerator and higher molecular weight protein amount in the denominator, a ratio value of about 10 to about 2000, and more usually about 120 to about 400, indicates that the subject from whom the sample was obtained likely had AD that the time the sample was taken. On the other hand, a ratio value of about 0.005 to about 15, and usually about 0.01 to about 1.2, and more usually about 0.015 to about 0.5, indicates that the subject from whom the sample was obtained likely did not have AD that the time the sample was taken.

It is to be understood that the value of the ratio varies with the pair of proteins chosen for the ratio. Thus, where albumin is used as the second proteinaceous material (B), and the about 90 kDa FLNA polypeptide fragment is (A), an A/B ratio of about 0.35 to about 1.5 indicates the subject had AD at the time the sample was taken, whereas an A/B ratio of about 0.003 to about 0.25 indicates the subject did not have AD at the time the sample was taken. Where (A) is as above, and GADPH is the second proteinaceous material (B), an A/B ratio of about 0.6 to about 2.1 indicates the subject had AD at the time the sample was taken, whereas an A/B ratio of about 0.005 to about 0.35 indicates the subject did not have AD at the time the sample was taken.

Similar ratios can be created using the about 280 kDa FLNA protein as the (A) material and another proteinaceous material present in the plasma or serum used to determine the ratio. A reference list of one or more such ratios can be prepared from a large number of subjects known to have or not have AD based on one or the other of the about 90 kDa FLNA polypeptide fragment and the about 280 kDa FLNA protein along with a second proteinaceous material present in the serum or plasma to be used for comparative purposes. The use of the about 90 kDa FLNA polypeptide fragment and the about 280 kDa FLNA protein is the particularly preferred ratio at this time.

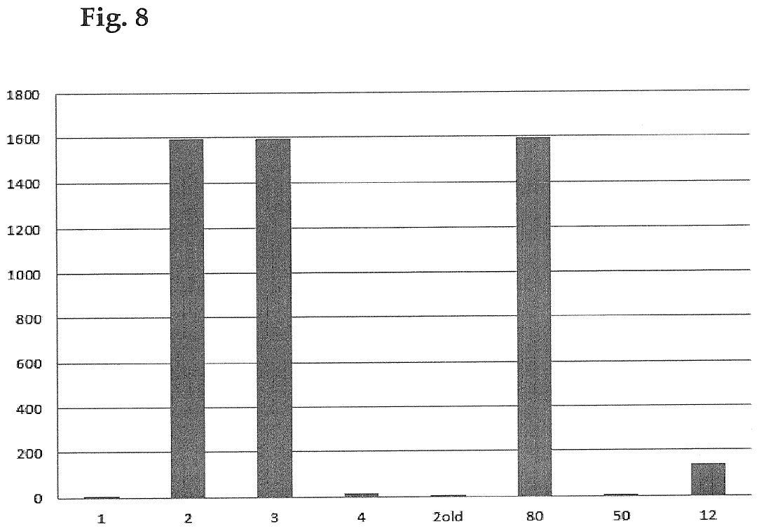

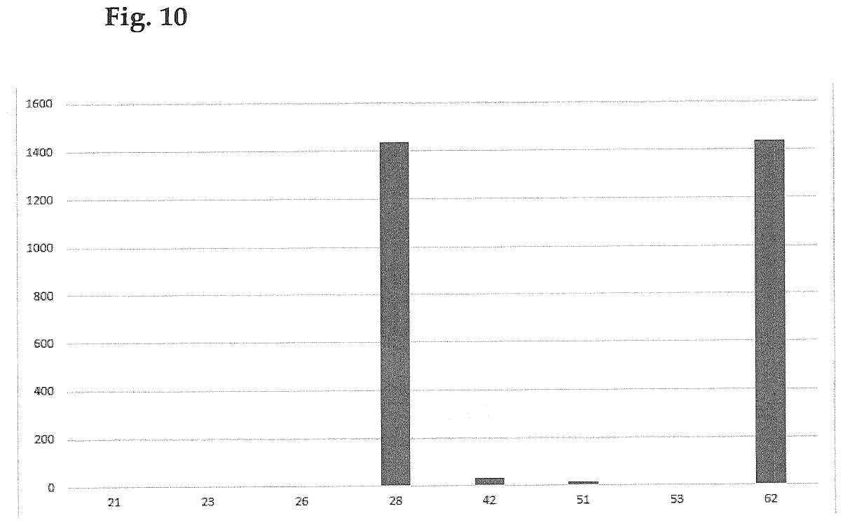

Results from samples from AD patients (AD) as diagnosed by PET scan or other technique and age-matched controls (AMC) are shown in Table 1, hereinafter, and in FIGS. 1-11. As will be seen from the data of Table 1 and in FIGS. 1-11, subjects that had AD at the time the sample was taken had large amounts of the about 90 kDa FLNA polypeptide fragment in the serum or plasma sample, and substantially no FLNA (about 280 kDa FLNA). Those results were reversed in age-matched cognitively normal controls; i.e., most subjects had no quantifiable about 90 kDa FLNA polypeptide fragment and large amounts of about 280 kDa FLNA protein.

Examining of the graphs of those figures, it is seen that although the magnitude of the amounts can vary greatly, the results obtained using the A/B ratios provide the similar results with less scatter. Those results of likely having AD or not are readily distinguishable from each other.

It can also occur that neither A nor B is present in a quantifiable amount as a result of a contemplated assay. When both of about 90 kDa FLNA polypeptide fragment and about 280 kDa FLNA protein are not present in a quantifiable amount (absent), an arbitrary amount is assigned that is about the same for both.

For example, arbitrary assignment of 50 and 60 are used herein for amounts of A and B when both are not present or are not quantifiable. Thus, the A/B ratio is 0.83 or 1.2 (50/60 or 60/50), or about 1. This result typically occurs with samples from young cognitively intact (YCI) subjects (preferably younger than 45 years old) that are free of AD symptoms. Results from assays of samples from such subjects are shown in Table 1 hereinafter.

As is well understood in science, presence and absence of a substance is not necessarily a yes or no answer to a question. Rather, as science progresses, that which was undetectable (or unquantifiable) yesterday may become so today or tomorrow or in 20 years.

Because of the march of science and technology, the absence of a before-discussed lower or higher molecular weight polypeptide/protein is hereby defined as the inability to detect or quantify that material using a published method that utilizes western blot technology and reagents commercially available in 2021 in the USA, and densitometric scanning provided by a GS-800™ calibrated densitometer, Bio-Rad Laboratories, Inc., Hercules, California, USA. The presence of one or both of the materials is similarly determined. Useful reagents and equipment for such analyses are discussed hereinafter.

In another preferred embodiment, the proteinaceous portions are separated by ultrafiltration that removes materials of a molecular mass greater than about 100 kDa from the sample, and the presence or absence of an about 90 kDa FLNA polypeptide fragment (phosphorylated or not) is determined using the ultrafiltrate, whose proteinaceous materials have masses of less than about 100 kDa. Full length FLNA (about 280 kDa FLNA protein) phosphorylated and not phosphorylated at position serine-2152 is among the separated proteinaceous molecules of molecular mass greater than about 100 kDa that are excluded from the ultrafiltrate and are present in the retentate. The full length FLNA has a molecular mass of about 280 kDa and is often referred to herein as FLNA, about 280 kDa FLNA, and pS²¹⁵²-FLNA for the 2152serine-phosphorylated molecule.

Thus, an assay that simply determines whether the pS²¹⁵²-90 kDa and/or about 90 kDa FLNA polypeptide is present in the ultrafiltrate can be utilized. More preferably, assays are carried out for both the lighter weight (about 90 kDa) FLNA polypeptide species and the heavier weight (about 280) FLNA protein.

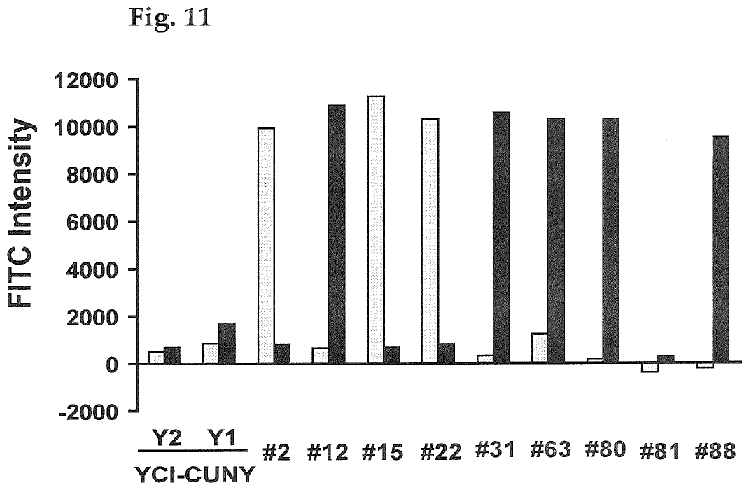

Results from one such assay are shown in FIG. 11. There, plasma samples were obtained and diluted 1 volume to 40 volumes with pH 7.4 Tris buffer. The resulting sample was divided into two portions. One portion was ultrafiltered using a Nanosep® 100 K OMEGA™ (P/N OD100C33) by Pall Life Sciences, Ann Arbor, MI, to remove proteinaceous materials having a molecular mass of greater than about 100 kDa.

An amount of the ultrafiltrate so prepared equal to about 0.25 or 0.5 □L of plasma was added to a well of a 96-well assay ELISA plate (filtered). An equal amount of the unfiltered plasma sample preparation was added to another well.

The well walls of the ELISA plate had previously been coated with strepavidin and were then coated with biotinylated rabbit monoclonal antibodies raised to the epitope containing the FLNA phosphorylated serine 2152 [rabbit monoclonal anti-pS²¹⁵²filamin A receptors [EP2310AY] Abcam® (cat #: ab75978)] as capture receptors. The filtered and unfiltered samples were added to their respective wells, proteins permitted to bind to the anti-pS²¹⁵² paratopes, and then the wells were rinsed again.

Mouse monoclonal antibodies to an epitope mapping between residues 9-27 from the N-terminus of FLNA (E-3; SC-17749, Santa Cruz Biotechnology, Inc.) were used to detect pS²¹⁵²-FLNA present and mouse anti-phosphoserine monoclonal antibodies [NeuroMab (UC Davis/NIH): Cat #: 73-292] were used to detect the pS²¹⁵²-90 kDa FLNA polypeptide. After permitting the proteins present to bind to the detection receptors, the wells were rinsed and then contacted with FITC-labeled anti-rabbit IgG to immunoreact with the mouse antibody-bound phosphorylated proteins. After rinsing, the fluorescences were determined from the wells and the results are shown in the graph of FIG. 11.

As is seen from a comparison of the diagnoses shown in FIG. 11 with those found in Table 1 for the same subjects, the two assays agreed completely.

### Assay Systems and Kits

FLNA is an abundant protein found in the peripheral circulation, and is processed upon platelets activation [Buitrago et al., bioRxiv 307397]. Buitrago et al. found that the onset of platelet activation results in FLNA cleavage to 100 and 90 kDa fragments. Because FLNA is abundantly present in platelets and the FLNA cleavage products readily release into the blood upon platelet activation, methods minimizing platelet activation should be used. These methods can include the use of clotting inhibitors such as sodium citrate/citric acid buffers, EDTA and similar chelators, avoiding aggressive centrifugation, and minimizing transit and storage time.

Still another embodiment of the present invention is a system for detecting the about pS²¹⁵²-90 kDa polypeptide and/or the about 90 kDa FLNA polypeptide (non-phosphorylated at serine 2152) present in a serum or plasma sample from a living subject, and thereby the likely presence or absence of Alzheimer's disease (AD) in that living subject at the time the sample for analysis was obtained.

One aspect of this embodiment comprises a contemplated system in kit form that includes a container holding capture receptor molecules such as antibodies or antibody paratope-containing portions that immunoreact with one or both of the about 90 kDa and/or pS²¹⁵²-90 kDa FLNA polypeptide to form a captured complex when that material is separated from any pS²¹⁵²-FLNA protein or about 280 kDa FLNA protein that may be present in an assayed sample. A label that indicates the formation of that capture complex such that the presence or absence of the capture complex correlates with presence or absence of the one or both of the about 90 kDa and/or about pS²¹⁵²-90 kDa FLNA polypeptide in the sample, and consequently the presence or absence of AD in the living subject whose blood sample was used.

A contemplated system in another aspect is a kit in which the recited elements are present packaged together. Thus, for example, capture receptor molecules are present in a separate package or container such as a vial. The above indicating label can also be in a separate package. Also, preferably included in such a kit are instructions for using those capture receptor molecules for the purpose of binding to the one or both of the about 90 kDa and/or about pS²¹⁵²-90 kDa FLNA polypeptide to form a capture complex.

Examples of containers include glass or plastic vials and multi-well plates that permit detection of one or both of the about 90 kDa and/or about pS²¹⁵²-90 kDa FLNA polypeptide in multiple samples. A contemplated kit can also contain, depending on the particular assay used, suitable labels as above and other packaged reagents and materials (e.g., wash buffers, and the like). Standard immunoassays, such as those described above, can be conducted using these kits.

A particularly preferred assay kit contains a packaged solid phase support whose assay surfaces are coated with paratope-containing capture receptor molecules that immunoreact with an epitope that is common to both the lower and higher molecular weight previously discussed proteinaceous FLNA materials. One illustrative example of such a capture antibody binds to an antigen (epitope) that includes phosphorylated serine 2152 of low and high molecular weight FLNA of interest here (anti-pS²¹⁵² receptor molecules) to form a capture complex.

Also present in a contemplated kit is a container holding first detection receptor molecules that selectively bind to captured phosphorylated and/or non-phosphorylated about 90 kDa FLNA polypeptide to form a captured complex. A label for detecting the presence of that captured low molecular weight FLNA-containing complex can be linked to or linkable to the first detection receptors. Another container holding second receptors that selectively bind to any captured phosphorylated and/or non-phosphorylated about 280 kDa FLNA protein molecule toward the N-terminus from the calpain H1 cleavage site (at about position 1761) along with a linked or linkable label for detecting the binding to the higher molecular weight FLNA protein molecule can also be present. The two labels utilized provide distinct signals so that the presence of both weights of filamin can be detected.

In a further contemplated system, another particularly preferred assay kit contains two or more solid phase supports whose assay surfaces are coated with paratope-containing capture receptor molecules that immunoreact with an epitope that is common to both the lower (about 90 kDa) and higher (about 280 kDa) molecular weight proteinaceous FLNA materials as discussed above, along with further containers as discussed below, and also includes instructions for use. The paratope-containing capture receptor molecules on the surfaces of the two or more solid phase supports of the kit are the same; i.e., have the same binding properties toward the FLNA molecules of both molecular weights.

One of this kit's containers includes first detection receptor molecules that specifically bind to both the lower (about 90 kDa) and higher (about 280 kDa) molecular weight proteinaceous FLNA materials when present in an aliquot of sample preparation. The detection receptors bind to a different region from that of the capture receptor molecules coated on the solid support. A detection receptor-linked or -linkable label that provides a signal indicating the total amount of both FLNA species present. When linked to the first detection receptor molecules, the label is usually in the container with those receptors. When linkable, the label molecules are typically in a separate container of the kit.

An above kit further contains a second container having second, detection receptors that bind specifically to only to the higher molecular weight FLNA molecules and another label as discussed before. Use of a second solid support as discussed above with a second sample preparation aliquot binds the same amounts of low and high molecular weight FLNA to the support. Reaction of the second, detection receptors and their labels, and determination of the amount present provides an amount of higher molecular weight FLNA molecules. That amount can be subtracted from the total amount obtained as discussed above to provide an amount of the lower molecular weight FLNA molecules. It is preferred that the labels used be the same for assaying the total amount of the possibly present high and lower molecular weight FLNA molecules so that different responses of different labels do not have to be dealt with.

Alternatively, the above kit further contains a second container having second, detection receptors that bind specifically to only to the lower molecular weight FLNA molecules and another label as discussed before. Use of a second solid support as discussed above with a second sample preparation aliquot binds the same amounts of low and high molecular weight FLNA to this support. Reaction of the second, receptors and their labels, and determination of the amount present provides an amount of lower molecular weight FLNA molecules. That amount can be subtracted from the total amount provides an amount of the higher molecular weight FLNA molecules.

Illustrative second, detection receptors that immunoreact with the higher molecular weight FLNA molecules are typically raised to portions of the molecule not present in the lower molecular weight polypeptide. Thus, an oligopeptide from the IgFLNa-1-15 or ABD regions of the molecule linked to a carrier molecule such as hepatitis B core antigen (HBsAg) or maleimide-activated keyhole limpet hemocyanin (KLH) can be used to induce production of antibodies that recognize the peptide sequence, and used to detect only the about 280 kDa FLNA protein molecule. The amino-terminus and carboxy-terminus of the about 90 kDa FLNA polypeptide each provide neo-epitopes that are not present in the higher molecular weight FLNA molecule and can be similarly linked to a carrier and used to induce antibodies that bind only to the about 90 kDa polypeptide. See, for example Birkett, U.S. Pat. No. 6,942,866 and the references therein.

In a counterpart system, the solid support is separately supplied and can be prepared by the user. Illustrative solid phase supports include multi-well plates that permit detection of the about 90 kDa FLNA polypeptide in multiple samples such as AcroPrep™ Advance filter plates available from Pall Life Sciences, or a microtiter plate available from Thermo Fisher Scientific, individual test tubes and particulate solids such as plastic beads such as those manufactured by Spherotech, Inc. of Lake Forest, IL, Firefly® particles of Abcam® Plc, and magnetic particles such as superparamagnetic polystyrene (SPP) particles sold as Dynabeads® M-280 Strepavidin by Thermo Fisher Scientific.

A contemplated kit can also include a label or an indicating means for signaling the presence of an immunoreaction between the detection receptor and the reacted protein in a capture complex. An indicating means permits the reaction product capture complex to be detected, and is packaged separately from the receptor when not linked directly to a detection receptor, as was noted previously.

More particularly, a kit can include one or more of the receptor molecules described herein, such as those that are used for detection of pS2152-phosphorylated or pS2152-non-phosphorylated anti-FLNA paratope-containing molecules, biotinylated paratope-containing molecules that bind to receptors of another species, and the like. Labelled receptor molecules that immunoreact with Fc portions illustratively include detecting molecules such as FITC-labeled anti-mouse Fc antibodies and HRP-conjugated anti-rabbit IgG, or other receptors well known to skilled workers that bind to receptors from non-self species.

Other optional components of a kit can include: buffers, such as pH 7.4 50 mM Tris used to dilute plasma or serum samples, buffers for use in carrying out the variously described immunoreactions and those useful for carrying out separations or denaturations as in SDS-PAGE analyses. The various components of the kit can be present in separate containers and certain compatible components can be pre-combined into a single container, as desired.

In addition to the above components, a kit can further include instructions for practicing the above-described methods. These instructions can be present in the kits in a variety of forms, one or more of which can be present in or on the kit. One form in which these instructions can be present as printed information on a suitable medium or substrate, e.g., a piece or pieces of paper on which the information is printed, in or on the packaging of the kit, in a package insert, etc. A further means is presence on a computer readable medium, e.g., diskette, CD, etc., on which the information has been recorded. Yet another means that can be present is a website address that can be used via the internet to access the information at a removed site. Any convenient means can be present in a kit.

In one preferred aspect, a contemplated kit contains an ELISA solid phase support such as a microtiter plate whose assay surfaces, e.g., well walls and bottoms, are coated with paratope-containing molecules that immunoreact with the epitope that includes phosphorylated or non-phosphorylated serine 2152 of FLNA as capture receptors, e.g., the rabbit monoclonal anti-pS²¹⁵² filamin A receptors [EP2310AY] Abcam® (cat #: ab75978) and anti-pS²¹⁵² filamin A rabbit polyclonal antibodies designated TA313881 and TA325463 of Origene Technologies, Inc. A particularly preferred multi-well plate is an AcroPrep™ Advance 96-well filter plate equipped with an Omega™ 100 kDa molecular mass cut off filter, available from Pall Life Sciences of Ann Arbor, MI.

Also included in a contemplated kit is a container of detection receptor molecules that react with a capture complex when present. One preferred such detection receptor molecules are paratope-containing molecules that immunoreact with mouse monoclonal anti-phosphorylated serine residue (anti-pS) receptors such as NeuroMab clone N259/48; UC Davis/NIH NeuroMab facility (Cat #:73-292).

The detection (anti-pS²¹⁵²) receptors are induced in an animal genus (genus 2; i.e., mouse) other than that used to induce the capture receptors (rabbit). The anti-pS²¹⁵² receptors can themselves be labeled, or labeled anti-genus 2 receptors can be used. Receptor molecules that immunoreact specifically with Fc portions of antibodies raised in other genera are commercially available and their use is illustrated herein.

In a particularly preferred kit, receptor molecules that specifically immunoreact with a antigenic (epitopic) site present in full length FLNA that is not present in a phosphorylated or non-phosphorylated 90 kDa FLNA polypeptide are used. These receptors are also preferably present in their own container.

One such contemplated receptor immunoreacts with a N-terminal FLNA sequence. Such receptors thus bind to a portion of the FLNA molecule between the N-terminus and N-terminus of repeat 16. Repeat 16 begins at position 1779 of the FLNA sequence of accession number P21333 in the UniProtKB/Swiss-Prot data base. These receptor molecules are used to detect a capture complex containing the phosphorylated or non-phosphorylated about 280 kDa FLNA protein species.

One illustrative detector receptor is a mouse monoclonal antibody that is sold as Filamin 1 (E-3):sc-177749 sold by Santa Cruz Biotechnology, Inc., that is reported in the product literature to be specific for an epitope mapping between amino acid residues 9-27 near the N-terminus of full length FLNA. Another illustrative detector molecule is FITC-linked naloxone (naloxone fluorescein #7315, Setareh Biotech, LLC; Eugene, OR) that binds to a site in repeat 24 near the C-terminus of full length FLNA.

As discussed above, it is preferred that these paratope-containing detection molecules (receptors) be induced in an animal genus (genus 2; i.e., mouse) other than that used to induce the capture receptors (rabbit). The anti-phosphorylated serine²¹⁵² (anti-pS²¹⁵²) FLNA receptors can themselves be labeled, or labeled anti-genus 2 receptors can be used.

Illustrative mouse monoclonal receptor molecules that immunoreact with a N-terminal FLNA sequence are discussed elsewhere herein and can be used in this kit. Such antibodies can carry a linked label or as label-linked paratope-containing molecules such as FITC-linked anti-mouse IgG can be used.

### Assays

In one embodiment, a serum or plasma sample from a living subject is collected and preferably maintained at a temperature of about 4 to about 10° C. prior to use in an assay. To that sample are added protease and phosphatase inhibitors. Illustrative commercially available protease and phosphatase inhibitors include complete, EDTA-free Protease Inhibitor Cocktail (Millipore Sigma catalog number 4693159001) and PhosSTOP™ (Millipore Sigma catalog number 4906845001). One tablet of protease inhibitor plus one tablet of phosphatase inhibitor are dissolved in 500 μL of double distilled water, votexed and approximately 53 μL added to each 1 ml of plasma or serum.

The sample is then preferably diluted to form a sample preparation, and is separated into its component proteinaceous materials. Illustrative methods of separation include by size and by charge. When separation is carried out by charge, the proteinaceous materials in the serum sample are separated according to their isoelectric point (pI), which is the pH value at which a particular molecule carries no net electrical charge. When separation is carried out by size, as is preferred, the proteinaceous materials are preferably separated electrophoretically, by ultrafiltration, or other means.

Once separated, the proteinaceous material is preferably immobilized and then contacted and reacted with an analyzing receptor. In this assay, only one receptor molecule that binds to a desired proteinaceous ligand is needed. For example, an antibody that binds to the phosphorylated FLNA serine2152 as discussed before and used illustratively in several examples herein can be used. That analyzing receptor can be linked to a labeling means or the labeling can be carried out separately as is well known as with fluorescent-labeled antibodies that bind to the Fc portion of the anti-phosphorylated FLNA serine2152.

Most biochemical laboratories have equipment for carrying out electrophoresis studies. Although such equipment is useful, standard gel electrophoresis equipment can be subject to user error and subjective bias. More modern equipment such as those machines sold under the names WES™, Peggy Sue™, Sally Sue™, and Simon™ available from the ProteinSimple division of Bio-Techne Corp. of Minneapolis, MN, can be used to provide enhanced speed, accuracy, reproducibility, and user subjectivity-free assays.

A second embodiment is adapted for an ELISA-type format and utilizes two different capture receptor molecules. Two microtiter plate sample wells or similar structures are preferably utilized per assay.

Two aliquots are taken from a preferably diluted plasma or serum sample preparation. Desired FLNA proteinaceous material of a first aliquot from a sample preparation is bound to first capture molecules that are themselves affixed to a first solid phase support. Illustrative solid phase supports are well known and include the inside surfaces of microtiter plate wells, magnetic beads as well as non-magnetic beads.

Using microtiter plate wells, which are preferred, as illustrative herein, the aliquot is placed into a first well, whose surfaces are coated with first capture molecules. Illustratively, those first capture receptor molecules bind to a region of FLNA that is to the N-terminal side of the calpain H1 cleavage site that occurs between FLNA repeats 15 and 16. As a consequence, the first capture receptor binds full length FLNA but does not bind to the about 90 kDa phosphorylated or non-phosphorylated polypeptide fragment.

After an appropriate maintenance time to permit binding of the FLNA species present in the sample to the capture molecules, the unbound sample is removed. The bound portion, when present, is typically rinsed. Unbound areas of the surface are often blocked from non-specific binding by treatment with an aqueous dispersion of bovine serum albumin or non-fat dry milk, as is well known. The amount of bound FLNA moieties is then determined using analyzing molecules as discussed below.

Exemplary first capture molecules include mouse monoclonal MAB1678 and goat pAb sc 7565. Exemplary analyzing (detecting) receptor molecules include rabbit pAb 75978 and rabbit pAb sc 130190, both of which bind to a sequence that includes the phosphorylated serine2152 (or 2151). Following removal of any unbound analyzing receptor molecules, an indicating means-containing receptor is reacted with the exemplary bound rabbit antibodies, any excess removed and the amount of full length FLNA moieties is determined.

The second well, whose surfaces are coated with second, different, capture receptor molecules, is utilized to determine the amount, if any, of serine2152-phosphorylated or non-phosphorylated about 90 kDa FLNA fragment that is present in the sample. Exemplary second capture receptor molecules include rabbit pAb sc-28284 (Santa Cruz) that bind near the C-terminus of FLNA. This capture receptor binds to both full length and the about 90 kDa phosphorylated FLNA molecules.

The total amount of FLNA full length and about 90 kDa fragment molecules is determined using analyzing molecules such as rabbit pAb 75978 (abcam Inc., Cambridge, MA) or rabbit pAb sc 130190 (Santa Cruz) that bind to an epitope that includes phosphorylated or non-phosphorylated serine2152 as discussed above. Preferably, the same analyzing receptor molecules are used in both portions of the assay (full length FLNA as well as full length and about 90 kDa polypeptide fragment) to minimize any errors that may be due to differences in binding avidity.

The amount of full length FLNA determined from the first well is subtracted from the total amount of FLNA species determined from the second well to obtain the amount of about 90 kDa FLNA polypeptide fragment present. The ratio of the about 90 kDa FLNA polypeptide fragment species to the amount of full length FLNA is determined, if desired.

It is preferred in this second embodiment that equal amounts of plasma or serum sample preparation are utilized in each well. If unequal volumes of the sample are used, the amount of numerator or denominator in the ratio is adjusted appropriately so that the resulting ratio is that which would have been determined from use of equal volumes.

### Results

### Western Blotting:

Illustrative studies were conducted in the laboratories of Dr. Haou-Yan Wang at CUNY School of Medicine, Manhattan, New York.

Frozen plasma (1 ml) derived from study subjects was removed from −80° C. freezer and placed on ice until completely thawed. The thawed plasma was then separated into ten 100 μl aliquots. Five μl of plasma was diluted 20-fold with 50 mM Tris HCl, pH 7.5 and then combined with 100 μl of SDS-PAGE sample preparation buffer containing 2-mercaptoethanol as reductant. The resultant solution was boiled for 5 minutes and then permitted to cool to room temperature for SDS-PAGE.

Twenty-five μl of the solubilized plasma was loaded onto 7.5% SDS-PAGE along with molecular weight marker (5 μl, protein ladder from ThermoFisher Scientific, Corp., Waltham, MA, USA) usually in 1^{st} left-hand lane. The protein samples were then size-fractionated under denaturing conditions according to manufacturer's specifics. The well-separated proteins were electrophorectically transferred to 0.2 μm nitrocellulose membrane (Bio-Rad Laboratories, Inc., Hercules, CA., USA) [300 mA, 2 hours].

The resultant nitrocellulose membranes were then washed three times (2 minutes each) with 0.1% Tween®-20 containing phosphate-buffered saline (PBS, pH 7.2) and then blocked at room temperature for 1 hour with 10% non-fat milk in 0.1% Tween®-20 containing PBS. The blots were washed three times (2 minutes each) with 0.1% Tween®-20 containing PBS and incubated with 1:1000 dilution of rabbit monoclonal anti-human-pS²¹⁵²FLNA antibodies [EP2310AY] (ab75978, Abcam® plc, Cambridge, UK) overnight (about 18 hours) at 4° C. The membranes were then washed 3 times (2 minutes each) with 0.1% Tween®-20 containing PBS, then incubated with 1:7500 HRP-conjugated anti-rabbit IgG (pre-adsorbed, from Santa Cruz Biotechnology, Inc., Dallas, TX, USA; and GE Lifesciences, Chicago, IL, USA, 1:1) for 1 hour at room temperature.

The membranes were again washed 3 times (1 minute each) with 0.1% Tween®-20 containing PBS and then once with distilled water for 1 minute. The immunoreactivity was detected by a chemiluminescent method (SuperSignal™ chemiluminescent reagents—Pierce/Thermo), and visualized by immediately exposing to X-ray film for 10-30 seconds (depending on the intensity of the signal). Specific protein bands were quantified by densitometric scanning (GS-800™ calibrated densitometer, Bio-Rad Laboratories). It is to be understood that density values so obtained are in arbitrary units and are not absolute in that longer exposure times can cause greater amounts of protein to be observed.

The blood samples were coded and the assays run blinded to the workers running them. Diagnoses of AD, and mild cognitive impairment (MCI) were separately and independently obtained using a PET-scanning technique such as the Amnyvid®, Vizamyl®, Neuraceq® and fluorodeoxyglucose assays along with clinical criteria. Results of these assays are shown in the Table 1 below.

TABLE 1
Assay Results*

| Subject ID | Age | DX | 90-kD | 280-kD | 90/280 | Albumin | GAPDH |
|---|---|---|---|---|---|---|---|
| 88 | — | AD | 6982 | 50 | 139.640 | 18397 | 10199 |
| 88 | — | AD | 7330 | 50 | 146.6 | 10979 | 8786 |
| 11 | 77 | AD | 6611 | 50 | 132.220 | 14323 | 9811 |
| 31 | 90 | AD | 9058 | 50 | 181.160 | 18289 | 9594 |
| 79 | 47 | AD | 8419 | 50 | 168.380 | 15881 | 9289 |
| 74 | 71 | AD | 8487 | 50 | 169.740 | 15908 | 9017 |
| 67 | 73 | AD | 8951 | 50 | 179.020 | 16463 | 11263 |
| 12 | 78 | AD | 8986 | 50 | 179.720 | 15825 | 9425 |
| 80 | 79 | AD | 9752 | 50 | 195.040 | 16601 | 9572 |
| 75 | 80 | AD | 7939 | 50 | 158.780 | 11665 | 8289 |
| 71 | 83 | AD | 8517 | 50 | 170.340 | 11765 | 8184 |
| A | — | AD | 14113 | 50 | 282.260 | 17851 | 8808 |
| 47 | 81 | AD | 13678 | 50 | 273.560 | 16473 | 8786 |
| 31 | 90 | AD | 12956 | 50 | 259.120 | 14506 | 8894 |
| 10 | 79 | AD | 17376 | 50 | 347.520 | 18499 | 9976 |
| 78 | 52 | AD | 17939 | 50 | 358.780 | 17110 | 8891 |
| 43 | 79 | AD | 14823 | 50 | 296.460 | 14078 | 8679 |
| 76 | 85 | AD | 13935 | 50 | 278.700 | 12448 | 8730 |
| B | — | AD | 19002 | 50 | 380.040 | 15311 | 9675 |
| 37 | 75 | AMC | 60 | 3955 | 0.015 | 19133 | 10432 |
| 73 | 78 | AMC | 60 | 4733 | 0.013 | 17601 | 10519 |
| 53 | 79 | AMC | 60 | 5355 | 0.011 | 16743 | 11614 |
| 68 | 76 | AMC | 121 | 7816 | 0.015 | 16491 | 9094 |
| 4 | 73 | AMC | 110 | 8835 | 0.012 | 12246 | 9482 |
| 2 | 80 | AMC | 122 | 4794 | 0.025 | 12956 | 9159 |
| 52 | 79 | AMC | 543 | 6656 | 0.082 | 13684 | 9682 |
| 50 | 74 | AMC | 1029 | 9602 | 0.107 | 19197 | 9968 |
| 33 | 67 | AMC | 724 | 9403 | 0.077 | 11918 | 9043 |
| 48 | 48 | AMC | 952 | 6717 | 0.142 | 15397 | 9581 |
| 26 | 75 | AMC | 1039 | 8581 | 0.121 | 13918 | 9561 |
| 7 | 82 | AMC | 1096 | 6907 | 0.159 | 13410 | 9977 |
| 22 | 73 | AMC | 1351 | 5039 | 0.268 | 15486 | 10080 |
| 15 | 71 | AMC | 1423 | 8577 | 0.166 | 14696 | 10724 |
| 36 | 67 | AMC | 1393 | 10023 | 0.139 | 14153 | 8985 |
| 54 | 73 | AMC | 1849 | 5389 | 0.343 | 15202 | 10807 |
| 27 | 74 | AMC | 1880 | 6203 | 0.303 | 13889 | 8282 |
| 46 | 68 | AMC | 4017 | 10293 | 0.390 | 17301 | 8994 |
| 41 | 77 | AMC | 3589 | 8338 | 0.430 | 13847 | 8486 |
| 34 | 80 | MCI | 9157 | 50 | 183.140 | 16497 | 10903 |
| | | AD | | | | | |
| 63 | 75 | MCI | 10957 | 50 | 219.140 | 12159 | 7994 |
| | | AD | | | | | |
| 29 | 67 | MCI | 60 | 4918 | 0.012 | 21088 | 9963 |
| | | NonAD | | | | | |
| 42 | 76 | MCI | 484 | 13038 | 0.037 | 19301 | 11016 |
| | | NonAD | | | | | |
| 44 | 80 | MCI | 723 | 14932 | 0.048 | 18134 | 9847 |
| | | NonAD | | | | | |
| 17 | 77 | MCI | 981 | 6262 | 0.157 | 16274 | 8953 |
| | | NonAD | | | | | |
| 38 | 82 | MCI | 2169 | 3646 | 0.596 | 15025 | 9153 |
| | | NonAD | | | | | |
| 44 | 80 | MCI | 3485 | 6643 | 0.525 | 14848 | 9970 |
| | | NonAD | | | | | |
| C | — | YCI | 60 | 50 | 1.200 | 21796 | 8862 |
| D | — | YCI | 60 | 50 | 1.200 | 17076 | 11624 |
| 81 | — | YCI | 60 | 50 | 1.200 | 11423 | 8404 |
| E | — | YCI | 60 | 50 | 1.200 | | |
| F | — | YCI | 60 | 50 | 1.200 | | |
| G | — | YCI | 60 | 50 | 1.200 | | |
| H | — | YCI | 60 | 50 | 1.200 | | |
| I | — | YCI | 60 | 50 | 1.200 | | |
| J | — | YCI | 60 | 50 | 1.200 | | |
| K | — | YCI | 60 | 50 | 1.200 | | |
| L | — | YCI | 427 | 50 | 8.540 | | |
| M | — | YCI | 4130 | 50 | 82.600 | | |
| N | — | YCI | 6407 | 50 | 128.140 | | |
| O | — | YCI | 5120 | 50 | 102.400 | | |

*Subject ID = identifying number or letter given to the subject at sample collection; Age = subject's age at time of collection; — = unknown age; DX = independent diagnosis; AD = Alzheimer's disease; AMC = age-matched control; MCI = mildly cognitively impaired; YCI = young cognitively intact; 90-kD = density reading for the about 90 kD phosphorylated band; 280-kD = density reading for the about 280 kD phosphorylated band; 90/280 = ratio of the density readings.

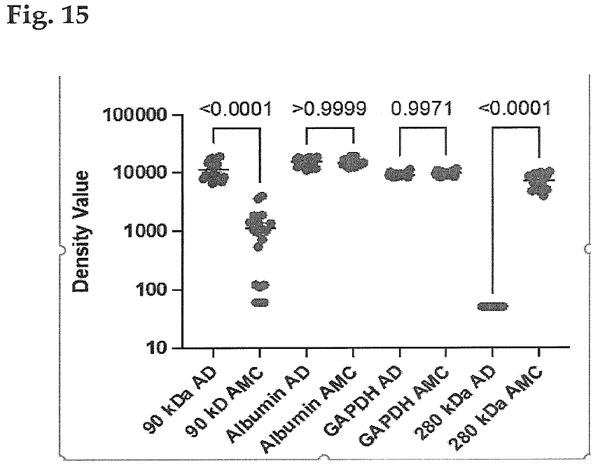

Similar density scans were also made for albumin and GAPDH that are present in the serum of each subject and are shown on the illustrative images of FIG. 1, FIG. 2 and FIG. 3 and in the two right-hand columns in Table 1. Density values for each of the about 90 kDa FLNA polypeptide fragment, Albumin, GAPDH and the about 280 kDa FLNA protein from serum samples of AD subjects and for the same proteinaceous materials from age-matched control sera are shown in FIG. 15.

As is seen, the values for Albumin and GAPDH are substantially the same for subjects with AD and their age-matched controls that do not have AD. To the contrary, values for each of the about 90 kDa FLNA polypeptide fragment and the about 280 kDa FLNA protein are different between those previously diagnosed with AD and those previously diagnosed as free of AD.

Thus, the densities for the about 90 kDa polypeptide FLNA fragment differ by about ten-fold between the subjects with AD and the age-matched controls. The density values for the about 280 kDa FLNA protein appear to differ by a factor of several thousand because zero about 280 kDa FLNA protein could be found for the AD subjects and an artificial value of 50 was used for these data.

Studies are presently underway to determine whether the about 280 kDa FLNA protein is truly absent, or its identity is being masked from the identifying antibodies by some presently unknown means. If that about 280 kDa FLNA protein is indeed absent from the serum or plasma of and subject, that absence can also be a biomarker for the subject having Alzheimer's disease.

Ratios of the densities of the about 90 kDa polypeptide FLNA fragment to Albumin, GAPDH and the about 280 kDa FLNA protein are shown in FIGS. 16A, 16B and 16C, respectively. Those density value ratios shown in both of FIGS. 16A and 16B were indicative of an Alzheimer's disease state for the AD subjects. However, as is seen, the ratio values were relatively similar among all subject types, making discrimination between them relatively difficult, but possible. To the contrary, FIG. 16C shows the differences between the AD subjects (AD and MCI-AD) and those without AD (AMC and MCI-nonAD) to be greater than about 100 fold, so that discrimination between subjects with and without AD was readily determined. The data for the young cognitively intact (YCI) subjects was spread over a wide range casting doubt on the accuracy of their diagnoses. Fortunately, those subjects do not often have AD.

Altogen Labs, Austin, Texas, USA, western blot (WB) procedure:

One μl of plasma for old samples was diluted with 50 mM Tris, pH 7.5 and then combined with Laemmli SDS-PAGE sample preparation buffer that contained 2-mercaptoethanol as reductant as discussed above. The resultant solution was boiled for 5 minutes and then allowed to cool down to room temperature for SDS-PAGE.

The sample was loaded onto 7% SDS-PAGE along with molecular weight marker (10 ml, protein ladder from ThermoFisher Scientific) usually in 1st left-hand lane. The protein samples were then size-fractionated under denaturing conditions according to manufacturer's specifics. The well-separated proteins were electrophorectically transferred to nitrocellulose membrane [300 mA, 2 hours].

The resultant membranes were then washed three times (5 minutes each) with 0.1% Tween®-20 containing phosphate-buffered saline (PBS, pH 7.2) and then blocked at room temperature for 1:15 hours with 5% non-fact milk in 0.1% Tween®-20 containing PBS. The blots were washed three times (5 minutes each) with 0.1% Tween®-20 containing PBS and incubated with 1:1000 dilution of anti-pS2152FLNA antibodies [EP2310AY] (ab75978, Abcam®) or pan-FLNA 1:2500 (Santa Cruz) for overnight (about 18 hours) at 4° C. The membranes were then washed 3 times (5 minutes each) with 0.1% Tween®-20 containing PBS, then incubated with 1:7500 HRP-conjugated anti-rabbit IgG (pre-adsorbed, from Santa Cruz Biotechnology and GE Lifesciences 1:1) for 1 hour at room temperature.

The membranes were again washed 3 times (5 minutes each) with 0.1% Tween®-20 containing PBS and then once with distilled water for 1 minute. The immunoreactivity was detected by a chemiluminescent method (SuperSignal™ chemiluminescent reagents—Pierce/Thermo), and visualized by immediately exposing to X-ray film for 30 seconds-2 minutes (depending on the intensity of the signal). Specific protein bands were quantified by densitometric scanning (GS-800 calibrated densitometer, Bio-Rad Laboratories) on ImageJ.

Reagent details are shown in the Table below:

| Entry | Item | Company | Catalog Number |
|---|---|---|---|
| 1 | Acryl/Bis Acryl 37.5:1 | Boston Bioproducts | BAC 30PA |
| 2 | TEMED | AMRESCO | 0761 |
| 3 | Kaleidoscope ™ | Bio-rad | 161-0737 |
| | prestained standards | | |
| 4 | Laemmli Buffer | Bio-rad | 161-0324 |
| 5 | PVDF Membrane | Bio-rad | 1610177 |
| | (0.2 μm) | | |
| 6 | Blotting Paper (7*10) | VWR | 28298-030 |
| 7 | Transfer Buffer | Bio-rad | 161-0734 |
| 8 | Dry Milk Powder | Research Products | M17200 |
| | | International Corp | |
| 9 | 20X PBS with | Teknova | P1176 |
| | 0.1% Tween ®-20 | | |
| 10 | anti-pS²¹⁵²FLNA | Abcam | ab75978 |
| | antibodies (rabbit) | | |
| 11 | Anti-Rabbit IgG | Santa Cruz | SC-2077 |
| 12 | Anti-Rabbit IgG pre- | GE Lifesciences | NA934V |
| | adsorbed | | |
| 13 | SuperSignal ECL WB | Pierce/Thermo | 32109 |
| | substrate | | |
| 14 | Developer Solution | CareStream Dental | 1901859/1900943 |
| 15 | Fixer | CareStream Dental | 901859/1901875 |
| 16 | X Ray Films | Thermo | 34093 |

Using the protocols noted above, Altogen Labs assayed four of the previous samples and four additional samples. The Alzheimer's disease status of each of the subjects had been previously determined (Indept. DX) and was withheld from the people conducting the assays. The results of that more limited assay are shown in Table 2 below.

TABLE 2
Altogen Labs Results

| Subject | Indept. DX | 90-Kda | 280-Kda | 90/280 | Assay DX |
|---|---|---|---|---|---|
| 1 | Normal | 79808.62 | 88785.42 | 0.898893 | Normal |
| 2 | AD | 79818.75 | 50 | 1596.375 | AD |
| 3 | AD | 79810.57 | 50 | 1596.211 | AD |
| 4 | Normal | 84912.98 | 4955.246 | 17.13598 | Normal |
| 2-old | Normal | 79817.14 | 88739.61 | 0.899453 | Normal |
| 80 | AD | 79794.66 | 50 | 1595.893 | AD |
| 50 | Normal | 79819.63 | 13140.8 | 6.074182 | Normal |
| 12 | AD | 6964.874 | 50 | 139.2975 | AD |

* Indept. DX = Independent diagnosis; Assay DX = Diagnosis from a present assay

A comparison of the results obtained by each group is shown in Table 3 below for the four repeated samples.

TABLE 3
Results Comparison

| Sub- | Altogen Assay | | | CUNY Assay | | |
|---|---|---|---|---|---|---|
| ject | 90-kD | 280-kD | 90/280 | 90-kD | 280-kD | 90/280 |
| Prior | 79817.14 | 88739.61 | 0.899453 | 122 | 4794 | 0.025 |
| 2 (2) | | | | | | |
| 80 | 79794.66 | 50 | 1595.893 | 9752 | 50 | 195.040 |
| 50 | 79819.63 | 13140.8 | 6.074182 | 1029 | 9602 | 0.107 |
| 12 | 6964.874 | 50 | 139.2975 | 9752 | 50 | 195.040 |

It is easily seen that although the density values obtained for the two phosphorylated proteins are different between the two assays, use of the ratio between the two phosphorylated protein western blot densities provided similar values and the same conclusions as to the Alzheimer's disease status of the subjects from whom the samples were obtained. This similarity of result indicates the robust character of this assay that is obtained from the use of density ratios of the two phosphorylated FLNA-related proteinaceous species, rather than densities of individual phosphorylated materials.

### Ultrafiltration

Illustrative studies were conducted in the laboratories of Dr. Haou-Yan Wang at CUNY School of Medicine, Manhattan, New York.

Plasma samples were diluted 1:40 with pH 7.4 Tris buffer and split into two portions. One portion was ultrafiltered through a Nanosep® 100 K OMEGA™ (P/N OD100C33) (PALL Corp., Ann Arbor, MI). This ultrafiltration device permits passage of molecules with molecular weights of about 100 kDa and less in the filtrate. The resulting filtrate and the unfiltered portion (retentate) were separately contacted with the walls of Reacti-Bind™ NeutrAvidin™ high binding capacity coated 96-well plate walls that had been coated with biotinylated antibodies that specifically react with avidin-labeled rabbit monoclonal antibodies to the phosphorylated serine 2152 of FLNA as capture molecules. The captured pS²¹⁵²-90 kDa FLNA polypeptide was detected using mouse anti-phosphoserine monoclonal antibodies [NeuroMab (UC Davis/NIH): Cat #: 73-292], whereas the captured pS²¹⁵²-FLNA was detected using mouse monoclonal IgG2a antibodies specific for an epitope mapping between amino acid residues 9-27 near the N-terminus of FLNA protein (SC-17749, Santa Cruz Biotechnology, Inc.). The relative amount of each immunoreactant was determined by reaction of each with FITC-labeled anti-mouse antibodies followed by excitation of the fluorescent label and measurement and plotting of the relative fluorescence intensities. The results of this study are shown in FIG. 11.

### Non-Phospho-Specific Receptors

A study was carried out using commercially available (OriGene, Inc.) phospho-specific polyclonal rabbit antibodies (TA325463) that immunoreact with an epitope that includes the phosphorylated 2152 serine residue of FLNA. A second study used mouse monoclonal antibodies (Antibody A) that were raised to an immunogenic polypeptide having an amino acid residue sequence that included phosphorylated serine at FLNA position 2152 as well as the native FLNA sequence on either side of that that residue.

A 10-mer peptide corresponding in sequence to positions 2148 through 2157 of the human FLNA UniProtKB/Swiss-Prot data base P21233 sequence was used as the immunogenic sequence. The serine residue at position 2152 of the peptide was phosphorylated. A cysteine amide residue was added at the C-terminus of that peptide for use in binding to maleimide-activated keyhole limpet hemocyanin (KLH) as the immunogenic carrier for the immunogen.

Two mouse hybridomas and two different monoclonal antibodies (A and B) were prepared from that immunization. The fine specificities of these molecules have not yet been identified. The first monoclonal [and hybridoma] (A) was used in the study discussed below.

Monoclonal antibodies A are IgG2 type. These antibodies specifically immunoreact with their immunizing peptide, and the about 90 kDa FLNA polypeptide fragment in western blot (WB) assays. No immunoreaction is seen with the phosphorylated about 280 kDa full length FLNA protein that is bound by the Origene rabbit polyclonal phospho-specific antibodies TA313881 and TA325463 in either western blot or ELISA assay formats.

Monoclonal antibodies B are IgM type. These antibodies specifically immunoreact with their immunizing peptide, and the about 90 kDa FLNA polypeptide fragment in western blot assays. No immunoreaction is seen with the phosphorylated about 280 kDa full length FLNA protein in a western blot format, but specific binding is found to the full length 2152-phosphorylated FLNA protein in an ELISA format.

We believe these antibodies have a unique profile distinct from all other antibodies directed to the region proximal to FLNA S2152 in that they only recognize the about 90 kDa FLNA polypeptide fragment and not the full length FLNA protein in a WB assay. This is not predictable as the epitope is present in both FLNA proteoforms, the about 90 kDa fragment and 280 kDa full length FLNA protein.

The samples were numbered and each was assayed by each antibody. Sample #88 from Table 1 that was obtained from a known AD patient was included as a positive control sample in each assay. Each antibody was used to identify the presence of the about 90 kDa FLNA polypeptide fragment and/or full length FLNA after SDS gel separation and visualization as described previously.

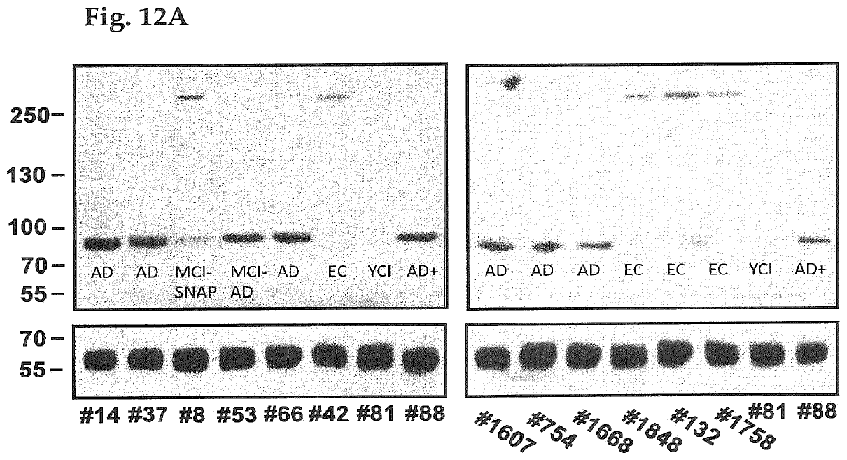

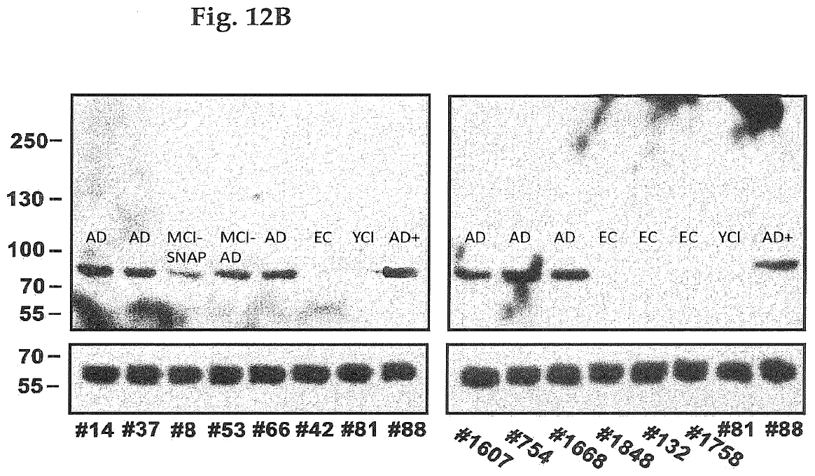

The results of this study are shown in FIG. 12A and FIG. 12B. As is seen, both antibodies bound to the about 90 kDa FLNA polypeptide fragment, but only the phospho-specific rabbit polyclonal antibodies bound to both the about 90 kDa FLNA polypeptide fragment and the full length, about 280 kDa FLNA protein. Both Antibody A and Antibody B accurately identified AD patients and clearly distinguished them from young cognitively intact (YCI) and elderly control (EC) subjects.

### Prognosis Assay

A contemplated assay can also be used prognostically assay the effectiveness of a treatment of a patient presumed to have Alzheimer's disease (AD). One medication is now approved for the treatment of AD by the United States Food and Drug Administration (FDA), aducanumab, sold as Aduhelm™, a monoclonal antibody. A small molecule medication now in clinical trials that has recently shown some efficacy in a small-scale clinical trial is a proprietary compound previously referred to as PTI-125, and now as simufilam, whose structural formula is shown below.

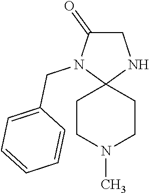 [SMILES: CN1CCC2(CC1)NCC(=O)N2Cc1ccccc1]

In accordance with a contemplated prognostic method, a living human patient is treated with a therapeutic composition containing an anti-AD effective amount of a medication (treating compound such as simufilam or aducanumab) or a pharmaceutically acceptable salt of that medication. Exemplary salts useful for a contemplated compound include but are not limited to the following: sulfate, bisulfate, hydrochloride, hydrobromide, acetate, adipate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemisulfate, heptanoate, hexanoate, fumarate, hydrochloride, hydrobromide, hydroiodide, 2-hydroxy-ethanesulfonate, lactate, maleate, methanesulfonate, nicotinate, 2-naphthalenesulfonate, palmoate, pectinate, persulfate, 3-phenyl-propionate, picrate, pivalate, propionate, succinate, tartrate, thiocyanate, tosylate, mesylate and undecanoate. Salts of the carboxylate group include sodium, potassium, magnesium, calcium, aluminum, ammonium, and the many substituted ammonium salts.

The reader is directed to Berge, J. Pharm. Sci. 1977 68(1):1-19 for lists of commonly used pharmaceutically acceptable acids and bases that form pharmaceutically acceptable salts with pharmaceutical compounds.

The amount of an about 90 kDa FLNA polypeptide fragment present in a sample preparation of serum or plasma from the blood of that living patient obtained before and after that treatment is determined. A later-determined amount of the about 90 kDa FLNA polypeptide fragment that is significantly less than an earlier determined amount of that about 90 kDa FLNA polypeptide fragment indicates that the medication engages its target and is effective in providing an improvement in the patient's disease state.

A small Phase 2a clinical trial that utilized a contemplated prognostic method along with other methodologies was first reported in the fall of 2019. The results of that trial were thereafter reported at Clinical Trials on Alzheimer's Disease (CTAD), December 4-7th, 2019 in San Diego, CA, and were published as Wang et al., J Prev Alzheimers Dis published 7 Feb. 2020. The use of the assay disclosed herein was neither shown nor discussed in that article.

Briefly, the study included 13 mild-to-moderate Alzheimer's disease patients, age 50-85, Mini Mental State Exam≥16 and ≤24 with a cerebrospinal fluid total tau/Aβ42 ratio 0.30. A pharmaceutical composition containing 100 mg of simufilam (PTI-125) in an orally administered tablet were given to the patients twice daily for 28 consecutive days. The study was conducted at five sites in the U.S. under an Investigational New Drug (IND) application.

Consistent with the drug's mechanism of action and preclinical data, the simufilam (PTI-125) reduced cerebrospinal fluid biomarkers of Alzheimer's disease pathology, neurodegeneration and neuroinflammation from baseline to Day 28. All patients showed a biomarker response to simufilam.

Total tau, neurogranin, and neurofilament light chain decreased by 20%, 32% and 22%, respectively. Phospho-tau (pT181) decreased 34%, evidence that the compound suppresses tau hyperphosphorylation induced by Aβ₄₂'s signaling through α7-nicotinic acetylcholine receptor. Cerebrospinal fluid biomarkers of neuroinflammation (YKL-40 and inflammatory cytokines) decreased by 5-14%. Biomarker effects were similar in plasma.

Aβ₄₂ increased slightly—a desirable result because low Aβ₄₂ indicates Alzheimer's disease. This increase is consistent with the compound's 1,000-fold reduction of Aβ₄₂'s femtomolar binding affinity to α7-nicotinic acetylcholine receptor.

Biomarker reductions were at least p 0.001 by paired “t” test. Target engagement was shown in lymphocytes by a shift in filamin A's conformation from aberrant to native: 93% was aberrant on Day 1 vs. 40% on Day 28. As a result, filamin A linkages with α7-nicotinic acetylcholine receptor and toll-like receptor 4, and Aβ₄₂ complexes with α7-nicotinic acetylcholine receptor and CD14, were all significantly reduced by the treatment.

The medication was safe and well-tolerated in all patients. Plasma half-life was 4.5 hours and approximately 30% drug accumulation was observed on Day 28 vs. Day 1.

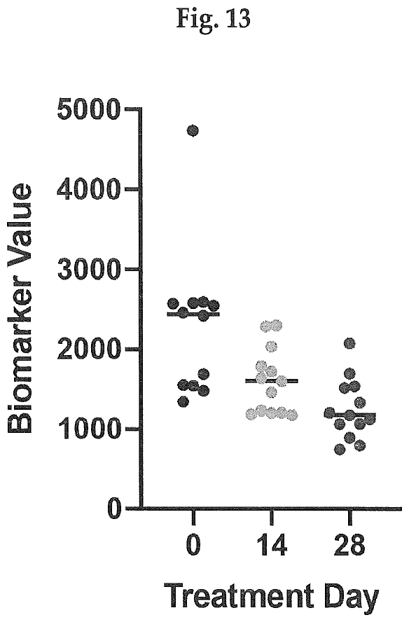

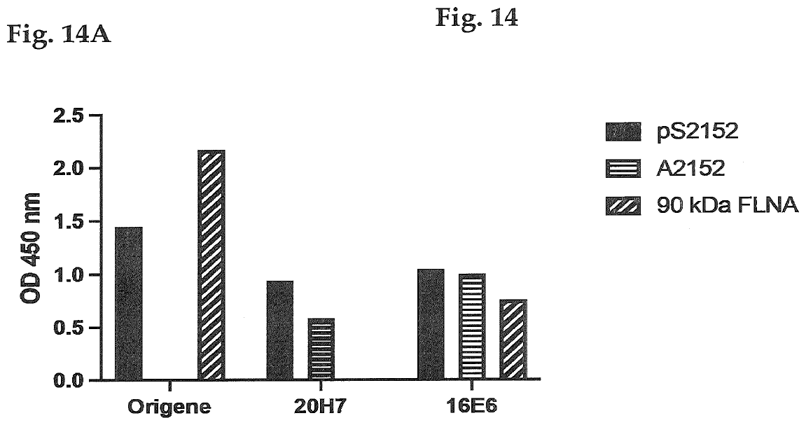

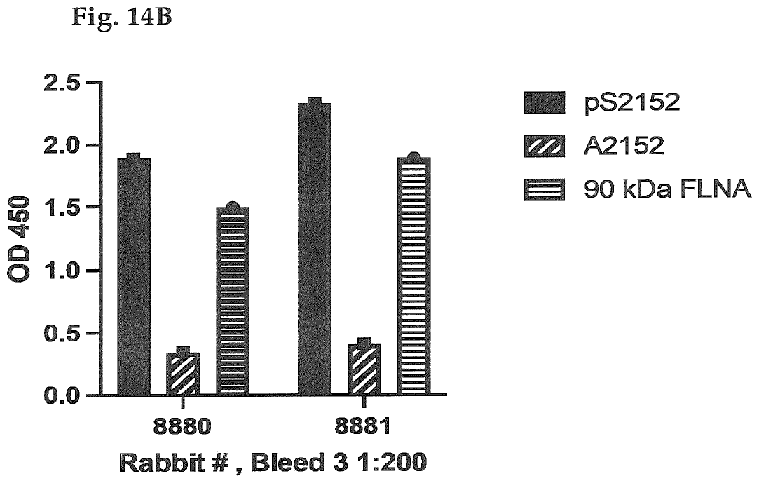

An assay for the presence and amount of the about 90 kDa FLNA polypeptide fragment in the plasma of each patient was also separately determined on days 0, 14 and 28 of the trial. The assay results for the group of 13 patients showed a decrease in the amount of the 90 kDa FLNA polypeptide fragment in the plasma at each of 14 and 28 days into the trial, P<0.01. Those results are shown graphically in FIG. 13 and indicate that the treatment appeared to be effective and was in agreement with the more standard assays.

Administration of the medication (treating compound) is typically carried out a plurality of times over a one-month period and can last through the remainder of the patient's life. This treatment regimen is exemplified by the twice daily administration of tablets containing 100 mg of simufilam (PTI-125) over a 28-day period.

The comparison of the presence and amount of the 90 kDa FLNA polypeptide is typically carried out a plurality of times each month in the first one to about 12 months and can be carried out less frequently once an effective medication and dosing regimen is determined.

The foregoing description and the examples are intended as illustrative and are not to be taken as limiting. Still other variations within the spirit and scope of this invention are possible and will readily present themselves to those skilled in the art.

1. A method of determining that a living human subject has Alzheimer's disease (AD) that comprises detecting the presence of an about 90 kDa polypeptide fragment of filamin A (FLNA) protein in an aqueous serum or plasma sample preparation that comprises serum or plasma sample obtained from said living human subject, which serum or plasma sample obtained from said living human subject may also contain FLNA protein, wherein said FLNA protein has a molecular mass of about 280 kDa and contains an amino-terminal actin binding portion bonded to 24 immunoglobin-like repeat domains (IgFLNa's) in the direction from amino-terminus to carboxy-terminus referred to as repeat 1 through repeat 24 (IgFLNa-1 through IgFLNa-24) and wherein said about 90 kDa FLNA polypeptide fragment includes the amino acid residue sequence of FLNA that includes repeats IgFLNa-16 through IgFLNa-23, the presence of said about 90 kDa FLNA polypeptide fragment in said sample indicating said living human subject has AD.

2. The method according to claim 1, wherein said about 90 kDa FLNA polypeptide fragment includes serine-2152 that is phosphorylated (pS²¹⁵²-90 kDa FLNA).

3. The method according to claim 1, wherein said about 90 kDa FLNA polypeptide fragment includes serine-2152 that is non-phosphorylated at FLNA serine-2152 (S²¹⁵²-90 kDa FLNA).

4. The method according to claim 1, wherein said detection of the presence of an about 90 kDa polypeptide fragment of filamin A (FLNA) protein is carried out by the steps of:
a) contacting said aqueous serum or plasma sample preparation with paratope-containing receptor molecules that immunoreact with said one or both of pS²¹⁵²-90 kDa and S²¹⁵²-90 kDa FLNA polypeptides to form a reaction mixture;
b) maintaining said reaction mixture for a time sufficient for said paratope-containing receptor molecules and said pS²¹⁵²-90 kDa and S²¹⁵²-90 kDa FLNA polypeptides and form an immunoreactant; and
c) detecting the presence or absence of said phosphorylated or non-phosphorylated about 90 kDa polypeptide fragment of FLNA from said immunoreactant.

5. The method according to claim 4, wherein the proteinaceous materials present in said aqueous serum or plasma sample preparation are separated into at least two portions prior to said contacting step (a), wherein said at least two portions include a first portion that may contain said pS²¹⁵²-90 kDa FLNA polypeptide fragment and a second portion that may contain said about 280 kDa FLNA protein.

6. The method according to claim 5, wherein said separation is carried out using reducing SDS-PAGE western blot analysis with a monomercaptan as the reducing agent.

7. The method according to claim 5, wherein said separation is carried out by ultrafiltration.

8. The method according to claim 7, wherein said ultrafiltration provides a first filtrate portion containing proteinaceous materials having a molecular mass of less than about 100 kDa, and a second retentate portion containing proteinaceous materials having a molecular mass of greater than about 100 kDa.

9. The method according to claim 5, wherein said separation is carried out by affinity binding of about 280 kDa FLNA molecules prior to detecting said about 90 kDa FLNA polypeptide fragment.

10. The method according to claim 4, wherein said paratope-containing receptor molecules specifically immunoreact with an epitope that includes the serine residue present at FLNA sequence position 2152 to form an immunoreactant binding product.

11. The method according to claim 4, wherein said paratope-containing receptor molecules are monoclonal antibodies.

12. A method of determining that a living human subject has Alzheimer's disease (AD) that comprises determining the ratio of an about 90 kDa polypeptide fragment of filamin A (FLNA) to a second proteinaceous material present in an aqueous serum or plasma sample preparation that comprises serum or plasma sample obtained from said living human subject that comprises:
a) separating proteinaceous materials present in said aqueous serum or plasma sample preparation into at least two portions, a first portion that contains said about 90 kDa FLNA polypeptide (A) when present and a second portion that contains said second proteinaceous material (B);
b) determining the relative amounts of said about 90 kDa FLNA polypeptide and said second proteinaceous material, determining the ratio of the two amounts, wherein an unquantifiably small amount of either A and/or B is assigned an arbitrary value of about 100^{th} of the amount of the quantifiable amount to avoid the presence of zero in a denominator or numerator, and
c) determining if the ratio obtained is within a predetermined A/B ratio value range for said about 90 kDa FLNA polypeptide and said second proteinaceous material obtained from subjects known to have AD and those known not to have AD, and thereby determining whether said human subject did or did not have AD at the time the blood was taken.

13. The method according to claim 12, wherein said second proteinaceous material is albumin, glyceraldehyde 3-phosphate dehydrogenase (GAPDH), or the about 280 kDa FLNA protein.

14. The method according to claim 13, wherein said second proteinaceous material is the about 280 kDa FLNA protein.

15. The method according to claim 14, wherein an A/B ratio of about 10 to about 2000, indicates that said living human subject likely had AD at the time the sample was obtained, whereas an A/B ratio of about 0.005 to about 15, indicates that said living human subject likely did not have AD at the time the sample was obtained.

16. A method of determining that a living human subject has Alzheimer's disease (AD) that comprises determining the ratio of an about 90 kDa polypeptide fragment of filamin A (FLNA) to about 280 kDa filamin A (FLNA) protein in an aqueous serum or plasma sample preparation that comprises serum or plasma sample obtained from said living human subject, that comprises:
a) separating proteinaceous materials present in said aqueous serum or plasma sample preparation into at least two portions, a first portion that contains said about 90 kDa FLNA polypeptide when present and a second portion that contains said about 280 kDa FLNA when present;
b) contacting said separated serum or plasma sample preparation portions with paratope-containing receptor molecules that immunoreact with said about 90 kDa FLNA polypeptide and said about 280 kDa FLNA or both when present to form a reaction mixture;
c) maintaining said reaction mixture for a time period sufficient for said paratope-containing receptor molecules and said about 90 kDa FLNA polypeptide when present and/or said about 280 kDa filamin A FLNA when present to form immunoreactant binding products A and B, respectively;
d) detecting and quantifying the relative amount of each of said immunoreactant binding products A and B, if any; and
e) determining the ratio of A/B, wherein an unquantifiably small amount of either A and/or B is assigned an arbitrary value of about 100^{th} of the amount of the quantifiable amount to avoid the presence of zero in a denominator or numerator, and wherein an A/B ratio of about 10 to about 2000, indicates that said living human subject likely had AD at the time the sample was obtained, whereas an A/B ratio of about 0.005 to about 5, indicates that said living human subject likely did not have AD at the time the sample was obtained.

17. The method according to claim 16, wherein said separation is carried out using reducing SDS-PAGE western blot analysis with a monomercaptan as the reducing agent.

18. The method according to claim 17, wherein said about 90 kDa FLNA polypeptide and said about 280 kDa FLNA are assayed from a single aliquot of said serum or plasma sample preparation.

19. The method according to claim 16, wherein said separation is carried out by ultrafiltration.

20. The method according to claim 19, wherein said ultrafiltration provides a first portion filtrate containing proteinaceous materials having a molecular mass of less than about 100 kDa, and a second portion retentate containing proteinaceous materials having a molecular mass of greater than about 100 kDa.

21. The method according to claim 16, wherein said separation is carried out by affinity binding about 280 kDa FLNA molecules.

22. The method according to claim 16, wherein said paratope-containing receptor molecules specifically immunoreact with an epitope that includes the serine residue present at FLNA sequence position 2152 to form an immunoreactant binding product.

23. A system for assaying for the likely presence of Alzheimer's disease (AD) in a living subject using an aqueous serum or plasma sample preparation comprising a serum or plasma sample obtained from that living subject that comprises:
a) a solid phase support whose assay surfaces are coated with paratope-containing capture receptor molecules that immunoreact with an epitope present on an about 90 kDa polypeptide fragment of filamin A (FLNA) to form an immunocomplex, wherein the FLNA protein has a molecular mass of about 280 kDa, contains an amino-terminal actin-binding portion bonded to 24 immunoglobin-like repeat domains (IgFLNa's) in the direction from amino-terminus to carboxy-terminus referred to as repeat 1 through repeat 24 (IgFLNa-1 through IgFLNa-24) and wherein the about 90 kDa FLNA polypeptide fragment comprises the amino acid residue sequence of FLNA that includes repeats IgFLNa-16 through IgFLNa-23;
b) a container holding first detection receptor molecules that bind to captured 90 kDa FLNA polypeptide fragment to form a captured complex; and
c) a label for detecting the presence of that captured complex.

24. The system according to claim 23, wherein said first detection receptor molecules immunoreact with an epitope that includes the serine residue present at amino acid residue position 2152, based on the full-length sequence of FLNA.

25. The system according to claim 23 further including a container holding second detection receptor molecules that react with a binding site present in about 280 kDa FLNA that is not present in said 90 kDa FLNA polypeptide fragment.

26. The system according to claim 25, wherein said second detection receptor molecules immunoreact with an epitope present in repeat 1 through repeat 15 (IgFLNa-1 through IgFLNa-15) amino acid residue sequence of FLNA, and is absent from repeats IgFLNa-16 through IgFLNa-23.

27. The system according to claim 25, wherein said first and second detection receptor molecules are Fc portion-containing antibody molecules raised in the same species of animal.

28. The system according to claim 27, wherein the animal species in which said first and second detection receptor molecules are raised is different from the species in which said capture receptors are raised.

29. The system according to claim 25 further including instructions for use.

30. The system according to claim 27, wherein said first and second detection receptor molecules are monoclonal antibodies.

31. The system according to claim 23, wherein the recited elements are present packaged together as a kit.

32. A method for determining the prognosis of treatment of a living human subject presumed to have Alzheimer's disease (AD) with a treating compound or a pharmaceutically acceptable salt of said treating compound that comprises the steps of:
(a) determining a first amount of an about 90 kDa polypeptide fragment of filamin A (FLNA) present in a first aqueous serum or plasma sample preparation comprising a serum or plasma sample obtained from that living human subject;
(b) treating said living human subject with a therapeutic composition containing an anti-AD effective amount of a treating compound or a pharmaceutically acceptable salt of that compound;
(c) determining a second amount of an about 90 kDa polypeptide fragment of FLNA in a second aqueous serum or plasma sample preparation from said human subject at a time at least about one month after the beginning of said treating; and
(d) comparing the amount of said about 90 kDa polypeptide fragment of FLNA present in sample preparations of serum or plasma obtained from the blood of said living patient before and after said treatment, wherein a later-determined amount that is significantly less than an earlier-determined amount is consistent with a prognosis of a benefit through use of the treatment to the patient from whom the samples were obtained.

33. The method according to claim 32, wherein the amounts of said about 90 kDa polypeptide fragment of FLNA present in said first and second sample preparations are determined as ratios relative to the amount of a second proteinaceous material that is present in human serum or plasma such that when the ratio of those two proteinaceous materials are compared before and after treatment as recited, a later-determined ratio amount that is significantly less than an earlier-determined ratio amount is consistent with a prognosis of a benefit through use of the treatment to the patient from whom the samples were obtained.

34. The method according to claim 33, wherein said second proteinaceous material is albumin, glyceraldehyde 3-phosphate dehydrogenase (GAPDH), or the about 280 kDa FLNA protein.

35. The method according to claim 33, wherein said second proteinaceous material is the about 280 kDa FLNA protein.

36. The method according to claim 33, wherein said about 90 kDa FLNA polypeptide is assayed from a single aliquot of said serum or plasma sample preparation.

37. The method according to claim 33, wherein said about 90 kDa FLNA polypeptide is separated from higher molecular weight materials possibly present in said serum or plasma sample preparation by ultrafiltration.

38. The method according to claim 37, wherein said ultrafiltration provides a first portion filtrate, containing proteinaceous materials having a molecular mass of less than about 100 kDa, and a second portion containing proteinaceous materials having a molecular mass of greater than about 100 kDa.

39. The method according to claim 32, wherein said treating step (a) is repeated a plurality of times during a one-month time period.

40. The method according to claim 32, wherein said comparing step (b) is repeated a plurality of times during a one-month period.

41. The method according to claim 32, wherein said anti-AD effective amount of a treating compound is aducanumab or simufilam, or a pharmaceutically acceptable salt of simufilam.

42. The method according to claim 32, wherein said anti-AD effective amount of a treating compound is simufilam, or a pharmaceutically acceptable salt thereof.
